(19) United States
(12) Patent Application Publication
(10) Pub. No.: US 2005/0033259 A1
(43) Pub. Date: Feb. 10, 2005

(54) Wall-mounted waste disposal system

(76) Inventor: David M. Stravitz, New York, NY (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/910,458

(22) Filed: Aug. 3, 2004

Related U.S. Application Data
(60) Provisional application No. 60/493,219, filed on Aug. 6, 2003.

(51) Int. Cl. A61F 13/15; A61B 19/00

(52) U.S. Cl. 604/403

(57) ABSTRACT

Waste disposal system including a housing defining a waste receptacle and including a waste-receiving slot into which waste is placed, one or more rolls of lining material and a roll forming mechanism for forming a roll of enclosed waste and the lining material with the waste being enclosed between windings of the lining material. In some embodiments, only a single roll of lining material is provided so that the waste is enclosed between successive windings of the lining material. In other embodiments, a second roll of lining material is provided and the waste is sandwiched between the lining materials from the two rolls. A mounting mechanism, such as one or more suction cups, may be attached to or integrated into the housing to enable the housing to be attached to a wall or other vertical surface.

### CROSS-REFERENCE TO RELATED APPLICATION

This application claims priority under 35 U.S.C. §119(e) of U.S. provisional patent application Ser. No. 60/493,219 filed Aug. 6, 2003, the entire contents of which are incorporated by reference herein.

### FIELD OF THE INVENTION

The present invention relates generally to waste disposal systems and more particularly to a wall-mountable waste disposal system for receiving and enclosing waste products in the form of a roll. The disposal system is particularly suited for the disposal of medical waste products which inherently pose a risk of infection to people handling the waste products.

### BACKGROUND OF THE INVENTION

Various types of containers for receiving medical waste have been developed to protect the users of such containers from possible infection by the waste products. Such medical waste products might include surgical sharps, such as needles, syringes, scalpel blades, gloves, gauzes, bandages and sponges, which could contain bodily fluids having potentially infectious germs. Containers for receiving such medical waste are often referred to as sharps containers and are typically designed to prevent the users from being accidentally cut or punctured by the waste contained therein.

The typical sharps container includes a base having a bottom and side walls defining a receptacle for receiving the medical waste and a lid removably attached to the top of the base. The lid usually includes an opening leading into the waste receptacle and a mechanism for selectively closing the opening so that waste can be inserted into the waste receptacle through the opening, e.g., by pressing the waste against the mechanism, and then the opening is closed to prevent the waste and odors emanating therefrom from being released from the container.

Generally, a bag lines the inside of the base of the sharps containers and receives the waste therein. When it is desired to remove the bag containing the waste from the sharps container, the lid is detached from the base (or vice versa) and the bag is lifted out of the base and tied. After the bag is lifted from the base and before it is tied, it is possible that the waste will fall out of the bag and the handler of the bag may be injured by the waste products in the bag.

It would therefore be desirable to design a sharps container in which the waste products are enclosed in a lining without any potential for causing injury by mishandling the waste products when the lining with enclosed waste products is handled during its removal from the container.

Other waste containers are specially designed to receive soiled diapers and are commonly referred to as diaper pails. A major concern with diaper pails is how to effectively confine odors emanating from the diapers. In one prior art diaper pail sold under the trademark “Diaper Genie”, a tubular core or cartridge houses a continuous length of flexible plastic tubing and the diapers are encapsulated in the tubing by forming a twist in the tubing above and below each diaper. There are several problems with diaper pails of this type as detailed in U.S. patent application Ser. No. 10/138,058, Ser. No. 10/456,428 and Ser. No. 10/693,087 by the same inventor and incorporated by reference herein. In addition, diaper pails of this type are not amenable to mounting on a wall or other vertical surface.

It would therefore be advantageous to have a wall-mountable waste disposal system suitable for diapers and other similar objects which encloses the diapers in a roll of material and therefore prevents the release of odors emanating from the diapers.

### OBJECTS AND SUMMARY OF THE INVENTION

It is an object of the present invention to provide a new and improved waste disposal system, especially one that can be mounted to a wall or other vertical surface.

It is another object of the present invention to provide a new and improved waste disposal system for receiving and enclosing waste products, in particular medical waste products.

It is yet another object of the present invention to provide a new and improved sharps container in which potentially infectious medical waste products are enclosed in a lining without any potential for causing injury during handling of the enclosed waste products when the lining with enclosed waste products is removed from the container.

It is still another object of the present invention to provide a wall-mountable waste disposal system suitable for diapers and other similar objects which encloses the diapers in a roll of material and therefore prevents the release of odors emanating from the diapers.

In order to achieve these objects and others, a waste disposal system in accordance with the invention generally comprises a housing defining a waste receptacle and including a waste-receiving slot into which waste is placed, a first roll of lining material and a roll forming mechanism for forming a roll of enclosed waste and the lining material with the waste being enclosed between windings of the lining material. In some embodiments, only a single roll of lining material is provided so that the waste is enclosed between successive windings of the lining material. In other embodiments, a second roll of lining material is provided and the waste is sandwiched between the lining materials from the two rolls. A mounting mechanism, such as one or more suction cups, may be attached to or integrated into the housing to enable the housing to be attached to a wall or other vertical surface.

The slot may divide the housing into an upper compartment and a lower compartment with the roll of lining material being arranged in the upper compartment and the roll of enclosed waste and lining material being formed in the lower compartment. An upper shelf of the housing supports the roll of lining material and may also define an upper wall of the slot. The upper shelf may be spaced from a rear wall of the housing to define a dispensing slot with the rear wall through which the lining material from the roll passes from the upper compartment to the lower compartment. In the alternative, a door for accessing the upper roll-receiving compartment may be provided in which case, the lining material from the roll passes through a dispensing slot formed between the upper shelf and the door and then passes through the waste-receiving slot.

When two rolls of lining material are provided, one may be arranged in the upper compartment as described above and another, lower roll may be rotatably mounted on a front wall of the housing by inserting a core of this roll into slots formed on brackets extending from the front wall. The lower roll is preferably mounted below the waste-receiving slot such that lining material from this roll passes over a lower shelf defining the waste-receiving slot as it unwinds. Waste is therefore carried on the lining material of this lower roll through the waste-receiving slot.

Instead of mounting the lower roll on the front wall of the housing, it may be housed in a dispensing compartment formed below the lower shelf defining the waste-receiving slot. A door is provided for accessing this dispensing compartment to enable replacement of the lower roll.

Several different constructions of the roll forming mechanism are envisioned. In one embodiment, a take-up roll is rotatably mounted in the housing and rotated by a rotation mechanism. The rotation mechanism engages with and supports the take-up roll at one axial end while the opposite axial end may be rotatably mounted and supported by a mounting projection. In one embodiment, the mounting projection is part of a spring-loaded release mechanism which includes a main body part having a knob at an outer end, the projection at the inner end, a flange for limiting axial movement of the main body part and a spring for biasing the projection inward into engagement with the axial end of the take-up roll. A locking device may be provided for preventing disengagement of the projection from the axial end of the take-up roll. The locking device can be designed to be released upon opening a door to the housing which enables access and removal of the roll of enclosed waste and lining material. In another embodiment, the mounting projection is part of a biasing member biased by a spring arm, e.g., a living hinge spring arm, to engage with and support the axial end of the take-up roll.

Several different constructions of rotation mechanisms are envisioned. In one embodiment, the rotation mechanism includes a manually actuatable handle and a transmission mechanism for converting the downward movement of the handle into rotational movement of the take-up roll while preventing upward movement of the handle from being converted into rotational movement of the take-up roll. The transmission mechanism includes a lever pivotable about a pivot axis upon manual actuation of the handle and at least one gear engaging with the lever for enabling the conversion of the manual actuation of the handle into rotation of the take-up roll. The lever has teeth formed on an outer surface thereof in engagement with the at least one gear. In another embodiment, the rotation mechanism includes a motor coupled to the housing and having an output shaft rotation upon actuation of the motor, a switch for actuating the motor and a coupling connected to the output shaft and to the take-up roll.

Various means for enabling attachment of the lining material of the roll or rolls to the take-up roll are contemplated. For example, a single section of adhesive may be used extending over a major part of an outer surface of the take-up roll. Instead, a plurality of discrete circular bands of adhesive may be used each extending at a different axial location around an outer surface of the take-up roll. In the alternative, an axially extending slot may be formed in the take-up roll allowing a full width of the lining material to pass therethrough. One or more barbs or nails may also be arranged on an outer surface of the take-up roll. In another construction, the take-up roll includes a tubular sleeve and plastic inserts separable from the sleeve and adapted to mount the sleeve in the housing. The sleeve includes adhesive for enabling attachment of the lining material of the roll or rolls thereto. In yet another construction, the take-up roll includes a disposable sleeve including adhesive for enabling attachment of the lining material of the roll or rolls to the sleeve and a reusable core adapted to mount the sleeve in the housing. The sleeve slides longitudinally onto and off of the core.

In some embodiments, the take-up roll can be arranged in a detachable bottom section which defines the waste-receiving compartment. The take-up roll includes a gear connected thereto which engages with the gear assembly described above when the bottom section is attached to a remaining part of the housing. The bottom section would also includes spring fingers for facilitating retention of waste being rolled onto the take-up roll. A hinged cover is provided on the bottom section and which is open when the bottom section is in engagement with a remaining part of the housing (and through which the lining material passes) and is closed when the bottom section is detached from the remaining part of the housing (to enclose the roll of waste and lining material in the bottom section).

The bottom section can be disposable or reusable by removing the roll of waste and lining material and inserting a new take-up roll. A service can be provided to pick-up used bottom sections for disposal or reuse, and provide new ones.

### BRIEF DESCRIPTION OF THE DRAWINGS

The invention, together with further objects and advantages thereof, may best be understood by reference to the following description taken in conjunction with the accompanying drawings, wherein like reference numerals identify like elements.

FIG. 1 is a perspective view of a first embodiment of a waste disposal system in accordance with the invention.

FIG. 2 is a cross-sectional front view taken along the line 2-2 of FIG. 1.

FIG. 3 is a cross-sectional side view taken along the line 3-3 of FIG. 2.

FIG. 4 is a cross-sectional side view taken along the line 4-4 of FIG. 2.

FIG. 5 is an exploded cross-sectional detail view of the rotation device for the take-up roll taken along the line 5-5 of FIG. 4.

FIG. 6 is a cross-sectional detail view of the retention mechanism of the take-up roll taken along the line 6-6 of FIG. 2.

FIG. 7 is a cross-sectional view similar to FIG. 6 but with the take-up roll in a released position.

FIG. 8 is a cross-sectional detail view of the retention mechanism of the take-up roll taken along the line 8-8 of FIG. 7.

FIG. 9 is a front elevational view showing the manner in which the take-up roll having a roll of waste enclosed by lining material is separated from the roll of lining material.

FIG. 10 is a fragmentary front elevational view showing the manner in which a new take-up roll is inserted into the waste disposal system.

FIG. 11 is a perspective view showing the manner in which a forward edge portion of a roll of lining material is attached to a take-up roll.

FIG. 12 is a perspective view of an alternate way to attach the forward edge portion of a roll of lining material to a take-up roll.

FIG. 13 is a perspective view of another way to attach the forward edge portion of a roll of lining material to a take-up roll.

FIG. 14 is an exploded perspective view of an alternate take-up roll and retention mechanism therefor.

FIG. 15 is an exploded perspective view of another alternate take-up roll.

FIG. 16 is a cross-sectional view taken along the line 16-16 of FIG. 15 but with the take-up roll in an assembled state.

FIG. 17 is a perspective view of a second embodiment of a waste disposal system in accordance with the invention.

FIG. 18 is a cross-sectional front view taken along the line 18-18 of FIG. 17.

FIG. 19 is a cross-sectional side view taken along the line 19-19 of FIG. 18.

FIG. 20 is a cross-sectional side view similar to FIG. 19 but with a new take-up roll in place.

FIG. 21 is a cross-sectional side view of an embodiment of a waste disposal system in accordance with the invention in which only a single roll of lining material is used.

FIG. 22 is a cross-sectional side view of another embodiment of a waste disposal system in accordance with the invention in which only a single roll of lining material is used.

FIG. 23 is a perspective view of another embodiment of a waste disposal system in accordance with the invention in which only a single roll of lining material is used.

FIG. 24 is a cross-sectional view taken along the line 24-24 of FIG. 23.

FIG. 25 is a cross-sectional view taken along the line 25-25 of FIG. 23.

FIG. 26 is a view of the rotation device for the take-up roll.

FIG. 27 is a perspective view of a bottom section of the housing of the waste disposal device shown in FIG. 23.

FIG. 28 is a cross-sectional view taken along the line 28-28 of FIG. 27.

FIG. 29 is a cross-sectional view taken along the line 29-29 of FIG. 27.

### DETAILED DESCRIPTION OF THE INVENTION

Referring to the accompanying drawings wherein like reference numerals refer to the same or similar elements, FIGS. 1-10 show a first embodiment of a waste disposal system 10 in accordance with the invention which generally comprises a housing 12 having a front wall 14 having a curved top portion 14a and a curved bottom portion 14b, a rear wall 16 having a curved top portion 16a and a curved bottom portion 16b and opposed side walls 18.

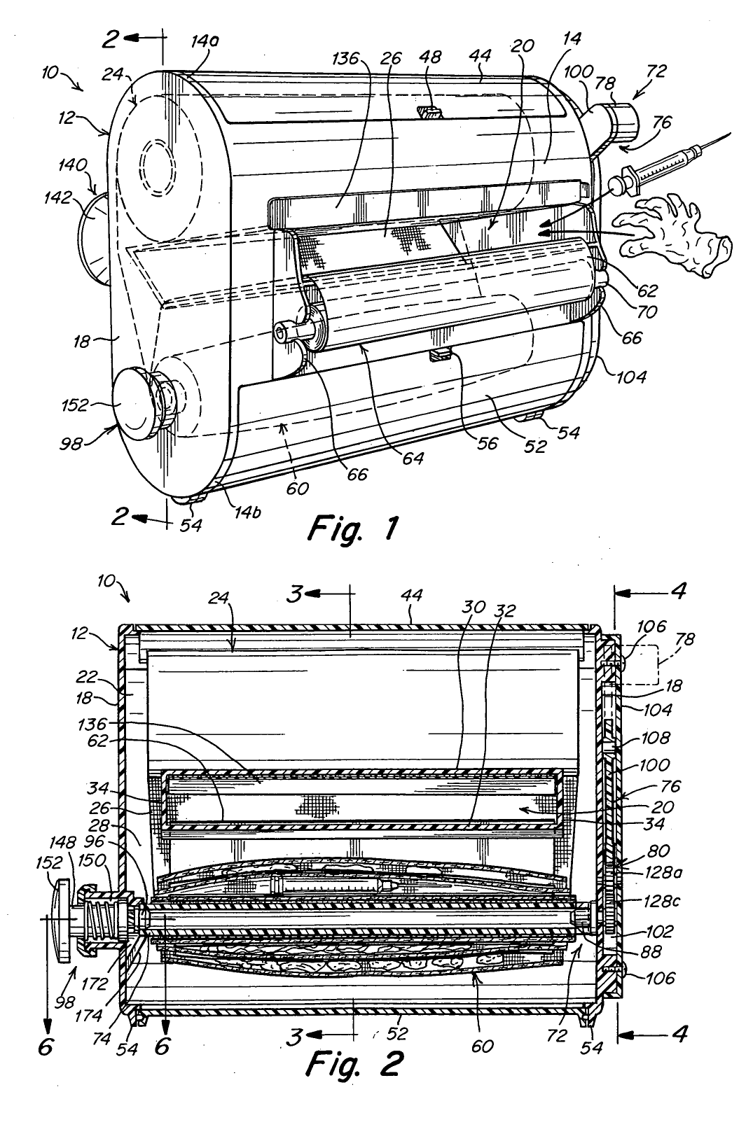

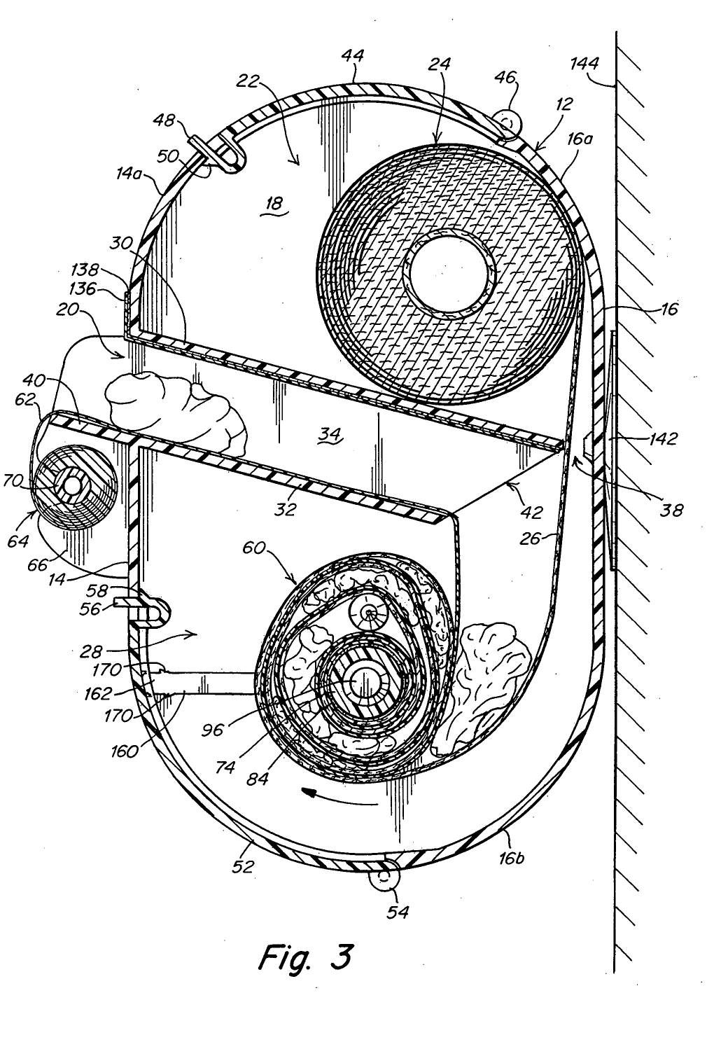

Housing 12 includes a horizontal chute or slot 20 into which waste products are placed and divides the interior of the housing 12 into an upper dispensing compartment 22 which receives a roll 24 of lining material 26 and a lower waste-receiving compartment 28 (see FIG. 3). The compartments 22,28 may be connected to one another on the sides of the slot 20 as shown in FIG. 2 if the slot 20 does not extend fully across the length of the housing 12.

Slot 20 is substantially rectangular and is defined by an upper shelf 30, a lower shelf 32 and opposed lateral walls 34. The upper shelf 30 extends inward into the interior of the housing 12 and downward. The upper shelf 30 supports the roll 24 of lining material 26 and the rear end of the upper shelf 30 is close enough to the rear wall 16 to prevent the roll 24 of lining material 26 from falling into the lower compartment 28, even when the lining material 26 is exhausted from the core 36 of the roll 24 (see FIG. 3). The lining material 26 passes through a dispensing slot 38 formed between the rear edge of the upper shelf 30 and the rear wall 16 to be situated inward of the inward end of the slot 20.

The lower shelf 32 also extends inward into the interior of the housing 12 and downward. A front lip 40 of the lower shelf 32 extends outward in front of the front wall 14 of the housing 12. The lower shelf 32 and upper shelf 30 may be parallel to one another or otherwise, e.g., angling toward one another at the inward ends. The rear edge of the lower shelf 32 is spaced from the rear wall 16 of the housing 12 more than the rear edge of the upper shelf 30, and the lateral walls 34 are angled between the rear edges of the upper and lower shelves 30,32, to provide the inward end of the slot 20 with an angled opening 42. The angled opening 42 facilitates the passage of waste from the slot 20 into the lower compartment 28.

The upper compartment 22 is accessible by means of a door or flap 44 pivotally connected to the curved top portion 16a of the rear wall 16 by hinges 46. The door 44 includes a resilient latch or clasp 48 engaging with the curved top portion 14a of the front wall 14. Pressing the latch 48 inward against its bias releases a shoulder 50 from engagement with the inner surface of the front wall 14 and then pulling the latch 48 away from the front wall 14 causes the door 44 to pivot about the hinges 46 and exposes the upper compartment 22. Other mechanisms for mounting and securing the door 44 to the housing 12 are also envisioned and within the scope of the invention. Access to the upper compartment 22 is necessary in order to insert replacement rolls 24 of lining material 26.

The lower waste-receiving compartment 28 is accessible by means a door or flap 52 pivotally connected to the curved bottom portion 16b of the rear wall 16 (or to the curved bottom portion 14a of the front wall 14) by hinges 54. The door 52 includes a resilient latch or clasp 56 engaging with the front wall 14. Pressing the latch 56 inward against its bias releases a shoulder 58 from engagement with the inner surface of the front wall 14 and then pulling the latch 56 away from the front wall 14 causes the door 52 to pivot about the hinge and exposes the lower compartment. Other mechanisms for mounting and securing the door 52 to the housing 12 are also envisioned and within the scope of the invention. Access to the lower compartment 28 is necessary in order to remove the roll 60 of waste enclosed by the lining material 26.

In the embodiment shown in FIGS. 1-10, the waste is enclosed or sandwiched between lining material 26 from the “upper” roll 24 and lining material 62 from another, “lower” roll 64. Sandwiching the waste between the lining material 26, 62 from the two rolls 24, 64 serves to trap the waste and prevent odors emanating from the waste from being released into the surrounding atmosphere.

The lower roll 64 of lining material 62 may be mounted on or stored in the housing 12 and is arranged to be situated on one side of the waste whereas the lining material 26 from the upper roll 24 is situated on the opposite side of the waste. As shown, the lower roll 64 of lining material 62 is rotatably mounted on the housing 12, specifically, on brackets 66 extending from the front wall 14 of the housing 12. The brackets 66 mount the lower roll 64 below the slot 20 so that as the lower roll 64 unwinds (in a clockwise direction), the lining material 62 passes over the lower shelf 32 through the slot 20. The width of the lining material 62 in the lower roll 64 may therefore be substantially the same as the width of the slot 20.

The brackets 66 may be integral with the front wall 14 as shown or formed separate from the housing 12 and fixed thereto. Each bracket 66 includes a slot 68 with a wide mouth, a middle portion narrower than a diameter of the core 70 of the lower roll 64 and a terminal portion wider than the diameter of the core 70 of the lower roll 64 to enable the lower roll 64 to rotate while its core 70 is positioned in the terminal portion. The core 70 of the lower roll 64 is longer than the width of the lining material 62. In view of the presence of the narrow middle portion, the core 70 of the lower roll 64 must be forced into the terminal portion of the slot 68 so that the lower roll 64 is securely retained in connection with the brackets 66 upon rotation of the lower roll 64. Other mechanisms for rotatably mounting the lower roll 64 on the housing 12 and preventing detachment of the lower roll 64 from the housing 12 upon its rotation can also be used in the invention.

A particular feature of this embodiment of the invention relates to the dimensioning of the width of the lining material 26 of the upper roll 24 relative to the width of the lining material 62 of the lower roll 64 to ensure that all waste in the slot 20, regardless of location, is enclosed by the lining material 26, 62 from both rolls 24, 64. That is, by providing the width of the lining material 26 of the upper roll 24 to be larger than the width of the slot 20, and thus larger than the width of the lining material 62 of the lower roll 64, any waste in the slot 20, including waste at the edges of the slot 20, is retained by the lining material 26 of the upper roll 24.

A mechanism 72 for forming the roll 60 of waste enclosed by lining material is arranged in connection with the housing 12. In the embodiment shown in FIGS. 1-10, the mechanism 72 comprises a take-up roll 74 rotatably mounted in the lower compartment 28 and a rotation device 76 for rotating the take-up roll 74. The rotation device 76 generally comprises a manually actuatable handle 78 and a transmission mechanism 80 for converting the downward movement of the handle 78 into rotational movement of the take-up roll 74 while preventing upward movement of the handle 78 from being converted into rotational movement of the take-up roll 74.

There are various different forms of the take-up roll 74 which may be used in the invention. Generally though, the take-up roll 74 is tubular and includes an attachment mechanism 82 to enable the lining material 26, 62 of the upper and lower rolls 24, 64 to be attached thereto. As shown in FIGS. 5 and 11, the attachment mechanism 82 may be adhesive 84 arranged on a major portion of the cylindrical outer surface of the take-up roll 74, i.e., a band of adhesive extending almost entirely between the axial ends of the take-up roll 74. Other take-up rolls 74 with various attachment mechanisms are described below with reference to FIGS. 12-16.

The transmission mechanism 80 of the rotation device 76 includes a member engaging with the take-up roll 74 for imparting rotational force to the take-up roll 74. To this end, the member of the transmission mechanism 80 is a tapered bayonet-type mounting projection 88 arranged along a side wall 18 of the housing 12 and engaging with an axial end 74a of the take-up roll 74 (see FIG. 5). The end 74a of the take-up roll 74 includes axially extending slots 90 which are designed to receive ribs 92 formed on the mounting projection 88. The ribs 92 extend outward from a central portion 94 of the mounting projection 88 which is designed to be inserted into the interior of the take-up roll 74. A washer may be arranged between the mounting projection 88 and the side wall 18 to increase friction between the take-up roll 74 and the side wall 18 and therefore prevent undesirable rotation of the take-up roll 74 relative to the side wall 18. Instead of a bayonet-type mounting projection 88 and corresponding axially extending slots 90 formed in the take-up roll 74, other mechanisms to provide rotational engagement between the mounting projection 88 and the take-up roll 74 can be applied in the invention.

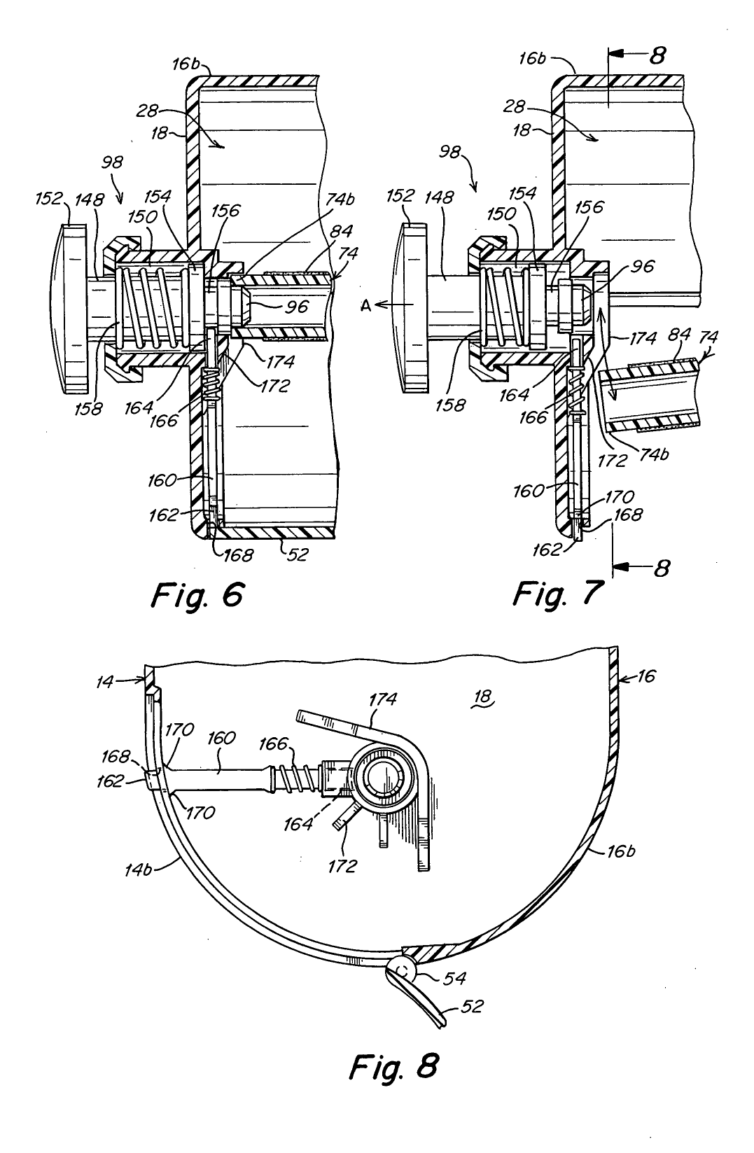

At the other axial end 74b, the take-up roll 74 is mounted onto a projection 96 formed integral with a spring-loaded release mechanism 98 which enables removal of the take-up roll 74 from its mounting in the lower compartment 28 (see FIG. 6). The take-up roll 74 is rotatable relative to the projection 96. Additional details about the release mechanism 98 are described below with reference to FIGS. 6-8.

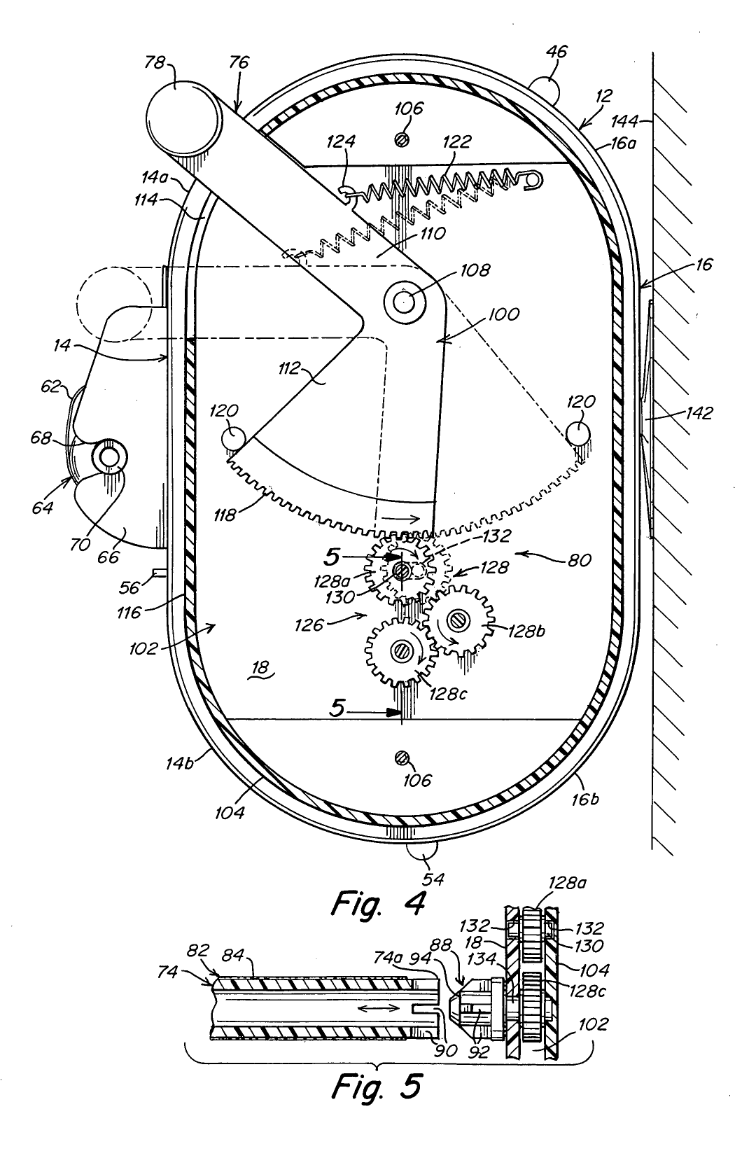

The transmission mechanism 80 of the rotation device 76 also comprises a lever 100 arranged substantially in a compartment 102 alongside the housing 12 defined between the side wall 18 and a cover plate 104 attached to the side wall 18 by an appropriate attachment mechanism, such as screws 106 and the like. The lever 100 pivots about a pivot pin 108 fixed to the side wall 18 and/or the cover plate 104 to thereby define a first lever arm 110 and a second lever arm 112. The cover plate 104 has essentially the same vertical cross-sectional shape as the housing 12, although smaller dimensions, and includes a slot 114 in a front wall 116 through which the first lever arm 110 extends (see FIG. 4).

The handle 78 is attached to or formed integral with the first lever arm 100. The second lever arm 112 includes a series of teeth 118 on an outer arcuate surface which may be formed as a rack gear attached to the lever 100 or formed integral therewith. To limit the movement of the first lever arm 110 in the slot 114, stops 120 are provided in the compartment and attached to the side wall 18 and/or the cover plate 104. To return the lever 100 to a position in which the first lever arm 110 is at or near the top of the slot 114 (and the handle 78 is at an upper position ready for use), a return spring 122 is mounted at one end to the side wall 18 and/or cover plate 104 and at the other end to a hook 124 formed on the first lever arm 110. Other mechanisms for biasing the lever 110 to return to a position in which the handle 78 is at an upper position can also be applied in the invention.

The transmission mechanism 80 also includes a gear system or assembly 126 including at least one gear for converting the pivoting movement of the lever 100 into rotation movement of the mounting projection 88 and thus rotation of the take-up roll 74. Specifically, the gear system 126 includes one-way clutch gears 128 which are designed to transmit the movement of the second lever arm 112 into rotation of the take-up roll 74 only when the second lever arm 112 rotates in a counterclockwise direction (in the embodiment shown in FIG. 4). Such a counterclockwise movement of the second lever arm 112 results when the handle 78 is pulled downward and the lever 100 is pivoted about the pivot pin 108 in a counterclockwise direction. When the handle 78 returns to its biased position, i.e., upward, and the lever 100 pivots in a clockwise direction, the gear system 126 does not transmit the pivoting movement of the lever 100 into rotation of the take-up roll 74.

To this end, the gear system 126 includes a first movable gear 128a having a rotation pin 130 mounted in elongate slots 132 formed in the side wall 18 and cover plate 104 (by movable, movement of the rotation axis of the gear 128a is meant). A rotation pin of a second gear 128b is mounted to the side wall 18 and cover plate 104 in a position in which the first gear 128a will engage it when the rotation pin 130 of the first gear 128a is situated at the rightmost position of the slot (in FIG. 4). The first gear 128a achieves this position when the lever 100 begins to rotate in the counterclockwise direction from the position shown in the solid lines in FIG. 4. Initially, the rotation pin 130 of the first gear 128a may be in the leftmost position in the slots 132. Upon counterclockwise rotation of the lever 100 about the pivot pin 108 from the position shown in solid lines in FIG. 4 (which results from the downward movement of the handle 78), the first gear 128a will rotate clockwise causing the rotation pin 130 thereof to move to the right in FIG. 4 until it is situated at the rightmost position in the slots 132. At this time, the first gear 128a will engage the second gear 128b and subsequent rotation of the lever 100 about the pivot pin 108 will cause clockwise rotation of the first gear 128a and counterclockwise rotation of the second gear 128b engaged therewith.

A rotation pin of a third gear 128c is mounted to the side wall 18 and cover plate 104 in a position in which it continuously engages the second gear 128b. The third gear 128c is connected to the mounting projection 88 (see FIG. 5) so that rotation of the third gear 128c is converted into rotation of the mounting projection 88 and thus rotation of the take-up roll 74. A shaft 134 extending through an aperture in the side wall 18 of the housing 12 may be used to connect the third gear 128c to the mounting projection 88. The mounting projection 88 may also be integrally formed with the third gear 128c.

Downward movement of the handle 78 is stopped when the first lever arm 110 contacts the cover plate 104 or when the second lever arm 112 contacts a stop 120. The rotation device 76 can be constructed so that these two events occur simultaneously.

Once the downward movement of the handle 78 is stopped and the person's grip on the handle 78 released, the handle 78 is moved upward. This is achieved by the compression of the return spring 122, which is tensioned during the downward movement of the handle 78 and the consequent counterclockwise rotation of the first lever arm 110, with the compression of the return spring 122 causing the lever 100 to rotate in a clockwise direction about the pivot pin 108 (from the position shown in dotted lines in FIG. 4). The rotation of the lever 100 in the clockwise direction is limited by contact between the first lever arm 110 and the cover plate 104 and/or by contact between the second lever arm 110 and one of the stops 120.

During the clockwise rotation of the lever 100, the second lever arm 112 also rotates in a clockwise direction and engages with the first gear 128a and causes the first gear 128a to rotate in a counterclockwise direction and move leftward from the position in which the rotation pin 130 thereof is situated in the rightmost position in the slots 132. As such, the first gear 128a is caused to disengage from the second gear 128b. Once the first gear 128a disengages from the second gear 128b, it will continue to rotate in a counterclockwise direction but such rotation is not transmitted to the second gear 128b.

A gear assembly 126 is thus provided in which the rotation of the lever 100 in one direction (caused by the downward movement of the handle 78) causes rotation of the take-up roll 74 while rotation of the lever 100 in the opposite direction (caused by the compression of the return spring 122) does not cause rotation of the take-up roll 74.

A shelf protector 136 is arranged partially on the outer surface of the front wall 14 and partially on the lower surface of the upper shelf 30 to prevent the accumulation of waste particles on the lower surface of the upper shelf 30 (see FIGS. 1 and 3). The shelf protector 136 may be disposable so that it can be replaced after a set period of time, e.g., once a day. In this case, the shelf protector 136 includes a mechanism for detachably mounting it to the outer surface of the front wall 14 and/or the lower surface of the upper shelf 30, e.g., adhesive 138. The replaceable shelf protectors 136 may thus include adhesive 138 on rear surfaces with a layer of coating material which is peeled off of the adhesive 138 upon placement of the shelf protector 136 in connection with the front wall 14 and/or upper shelf 30.

A mounting mechanism 140 is provided for mounting the housing 12 to a wall or other vertical surface 144. In the illustrated embodiment, the mounting mechanism 140 is a plurality of suction cups 142 fixed to the rear wall 16 of the housing 12, with the suction cups 142 extending through the rear wall 16 and adhering to the wall 144 via suction as shown in FIG. 3. Instead of several suction cups, a single large suction cup can be provided. Other types of mounting devices, such as brackets, can also be fixed to the housing 12 and then attached to the wall or other vertical surface 144 in order to mount the housing 12 to the wall or other vertical surface 144.

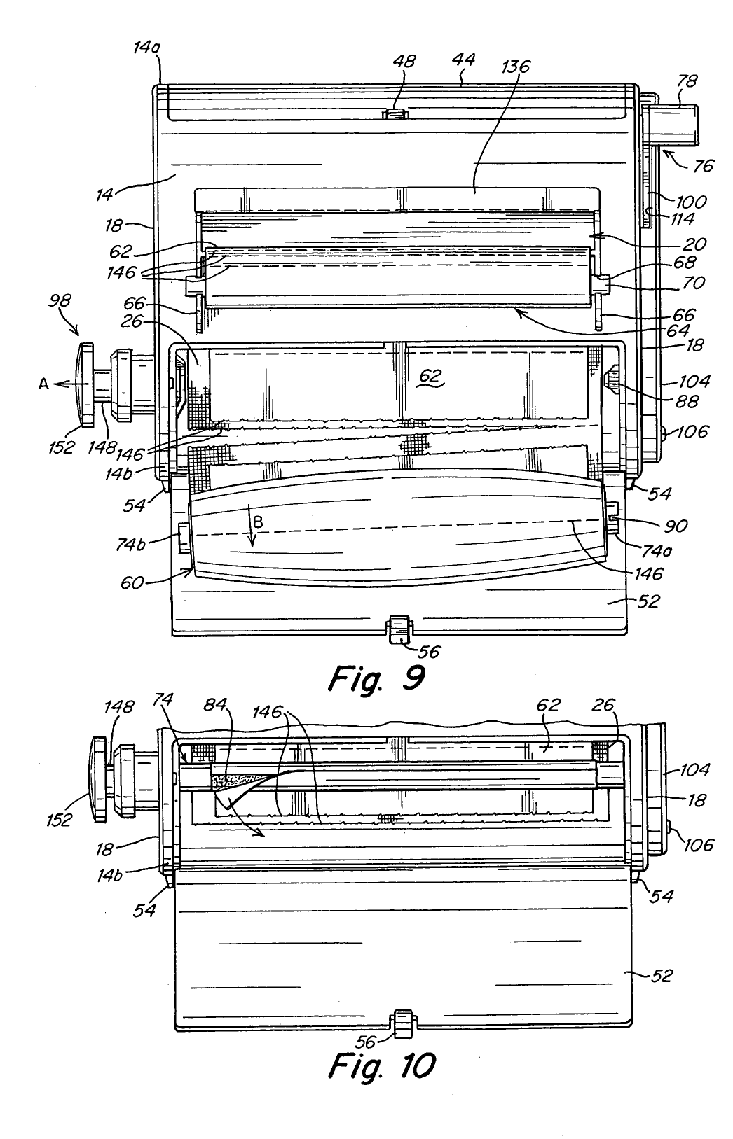

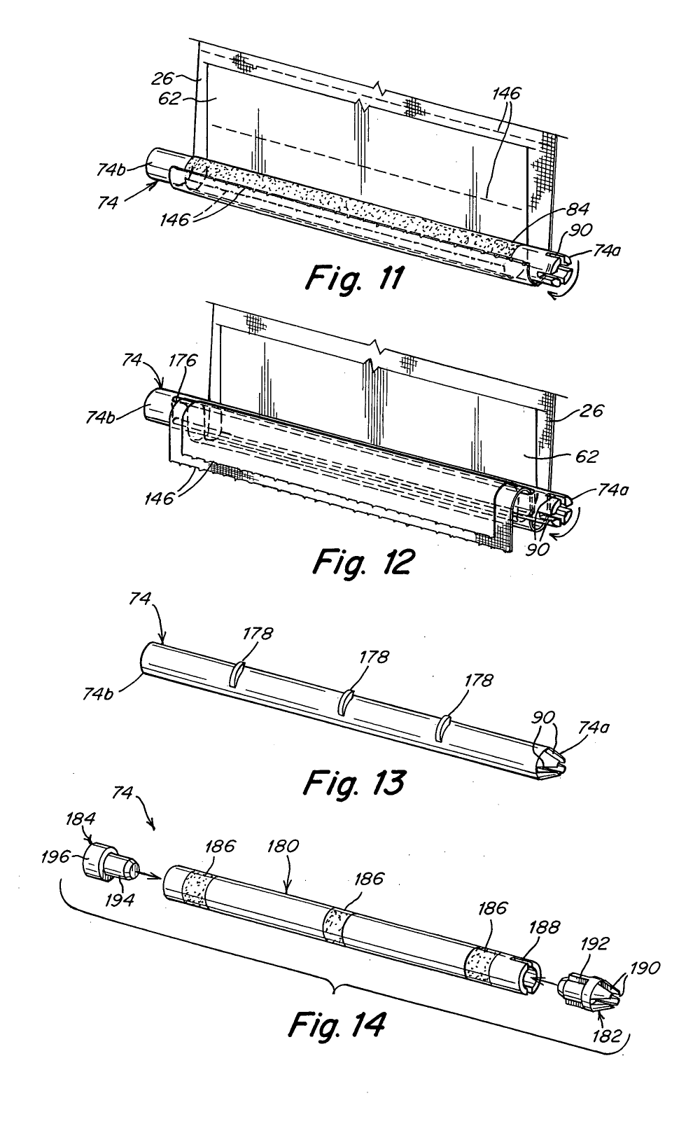

The lining material 26, 62 in the embodiments described above may be of any type of plastic film or of fibrous material such as paper, or a coated fibrous material, e.g., plastic coated paper. In one embodiment, the lining material 26 of the upper roll 24 is paper coated with plastic (with the paper side contacting the waste) and the lining material 62 of the lower roll 64 is plastic film. Also, the lining materials 26, 62 may be provided with perforation lines 146 extending between the lateral edges which enable the lining material 26, 62 to be easily torn and therefore used to form multiple rolls of enclosed waste and lining material (see FIGS. 9 and 10). The longitudinal spacing between the perforation lines 146 may be selected as desired.

Referring now to FIGS. 6-8, the release mechanism 98 includes a substantially cylindrical main body part 148 received in a compartment 150 formed by the housing 12 along a side wall 18. The main body part 148 has a knob 152 at an outer end and the projection 96 at the inner end. The axial end 74b of the take-up roll 74 is designed to fit over the projection 96. A flange 154 is formed on the main body part 148 and arranged in the compartment 150. Movement of the flange 154 is limited by the construction of the compartment 150 and in turn, axial movement of the release mechanism 98 in the direction of arrow A is limited by the permissible movement of the flange 154 (see FIG. 7). An annular groove 156 is arranged between the flange 154 and the projection 96. A compression spring 158 is interposed between the flange 154 and the outer wall of the compartment 150 and biased to press the flange 154 inward.

The release mechanism 98 is designed to enable rotation of the take-up roll 74 about the projection 96, and prevent inadvertent removal of the take-up roll 74 from its mounting location. To this end, as shown in FIGS. 6 and 8, a lock-out arm 160 is arranged in the housing 12 and includes a door-engaging tab 162 at one end, which engages with the door 52 to the lower compartment 28, and locking projection 164 at the other end. A compression spring 166 is arranged on the lock-out arm 160 to bias the locking projection 164 into the annular groove 156. When the locking projection 164 is situated in the annular groove 156, axial movement of the release mechanism 98 is prevented and thus the projection 96 is fixed in a position in which the take-up roll 74 is secured thereon. The door-engaging tab 162 is movable in a slot 168 formed in the housing 12 and projections 170 are formed on the lock-out arm 160 proximate the door-engaging tab 162 to limit movement of the lock-out arm 160 through the slot 168 (see FIGS. 7 and 8).

When the door 52 is opened, the compression spring 166 expands to urge the lock-out arm 160 outward and the door-engaging tab 162 thereof is urged through the slot 168 until the projections 170 engage the housing 12. As a result, the locking projection 164 is removed from the annular groove 156. Once the locking projection 164 is removed from the annular groove 156, the release mechanism 98 is movable in the axial direction (arrow A) by pulling the knob 152 away from the housing (see FIG. 7). When the release mechanism 98 is moved in the axial direction, the projection 96 is moved out of engagement with the take-up roll 74 enabling the take-up roll 74 to be removed from the lower compartment 28 and replaced. Thus, it is necessary to open the door 52 in order to remove the take-up roll 74. If it is attempted to pull the knob 152 away from the housing 12, so long as the door 52 is closed, the release mechanism 98 will not move outward and thus the take-up roll 74 will remain secured between the projection 96 on the release mechanism 98 and the mounting projection 88.

To assist in placement of a new take-up roll 74 into position between the projection 96 on the release mechanism 98 and the mounting projection 88, angled guide ramps 172 and a locating rim 174 are formed on the inner surface of the side wall 18 on which the release mechanism 98 is arranged (see FIG. 8). The guide ramps 172 serve to push the new take-up roll 74 toward the mounting projection 88, when the end 74b of the take-up roll 74 to be mounted on the projection 96 of the release mechanism 98 engages with the guide ramps 172, and assist in aligning the take-up roll 74. The locating rim 174 extends further inward than the guide ramps 172 and also serves to facilitate the proper placement of the end of the take-up roll 74 on the projection 96 of the release mechanism 98. The locating rim 174 has an arcuate shape with the center being in a position in which the end 74b of the take-up roll 74 will receive the projection 96 of the release mechanism 98 when the take-up roll 74 contacts the center (and upon release of the outward force being applied to the release mechanism 98 which causes the compression spring 158 to urge the projection 96 into the end 74b of the take-up roll 74).

It is desirable to provide the guide ramps 172 and locating rim 174, or other suitable positioning mechanism, on the same side of the housing 12 as the release mechanism 98 because the release mechanism 98 provides the primary axial play for placing a take-up roll 74 in position, by virtue of the presence of the compression spring 158, and not the mounting projection 88 on the opposite side wall 18 of the housing 12. Thus, installing or replacing the take-up roll 74 would entail first pulling the release mechanism 98 axially outward (or maintaining it in an axially extended position), placing the end 74a of the take-up roll 74 with the axially extending slots 90 on the mounting projection 88, then guiding the other end 74b of the take-up roll 74 over the guide ramps 172 and along the locating rim 174 and after the end 74b of the take-up roll 74 is centered along the locating rim 174, releasing the outward pulling of the release mechanism 98 to cause the release mechanism 98 to move inward and the projection 96 to pass into the end 74b of the take-up roll 74.

To prepare the device 10 for use, the housing 12 is mounted to a wall 144 by pressing the housing 12 against the wall 144 to create a suction force via the suction cups 142. The doors 44, 52 are opened and the upper roll 24 is placed into the upper compartment 22 and unwound slightly until a forward edge portion can be guided through the dispensing slot 38 between the upper shelf 30 and the rear wall 16 of the housing 12 and pulled downward from the open lower compartment 28. The lower roll 64 is mounted onto the mounting brackets 66 and unwound slightly and a forward edge portion is forced through the slot 20 until it can be pulled downward from the lower compartment 28. In this position, the forward edge portion of the lining material 26, 62 of both the upper and lower rolls 24, 64 is situated in the lower compartment 28.

A take-up roll 74 is mounted in the lower compartment 28 of the housing 12, either before, during or after the upper roll 24 is placed into the upper compartment 22 and the lower roll 64 is mounted to the housing 12 and the forward edge portions of the lining material 26, 62 thereof are fed into the lower compartment 28. Mounting of the take-up roll 74 involves pulling the release mechanism 98 axially outward, placing the end 74a of the take-up roll 74 with the axially extending slots 90 on the mounting projection 88 such that the slots 90 received the ribs 92 on the mounting projection 88, then guiding the other end 74b of the take-up roll 74 over the guide ramps 172 and along the locating rim 174 and after the end 74b of the take-up roll 74 is centered along the locating rim 174, releasing the outward pulling of the release mechanism 98 to cause the release mechanism 98 to move inward and the projection 96 to pass into the end 74b of the take-up roll 74.

The forward edge portions of the lining material 26, 62 of the upper and lower rolls 24, 64 are then adhered to the take-up roll 74. Specifically, the adhesive 84 on the take-up roll 74 is exposed by removing a peel strip, if not previously exposed, and the forward edge portion of the lining material 62 of the lower roll 64 is adhered to the adhesive 84. The forward edge portion of the lining material 26 of the upper roll 24 is then adhered to the adhesive 84. The doors 44, 52 are closed and the device 10 is ready for use. If an optional shelf protector 136 is not present, one may be attached to the lower surface of the upper shelf 30 by removing the coating material from the adhesive 138 and adhering the shelf protector 136 to the lower surface of the upper shelf 30.

In one exemplifying use, waste, such as a syringe and a glove, is placed into the slot 20 onto the lining material 62 from the lower roll 64 (see FIGS. 1 and 3). In view of the downward inclination of the lower shelf 32, the waste does not fall forward out of the slot 20. The handle 78 is then manually grasped and pulled downward causing rotation of the second lever arm 112 in the counterclockwise direction and attendant movement of the rotation pin 130 of the first gear 128a rightward in the slots 132 until it engages the second gear 128b. The continued rotation of the second lever arm 112 causes continued rotation of the first gear 128a which in turn causes rotation of the now-engaged second gear 128b which in turn causes rotation of the third gear 128c and the mounting projection 88. Rotation of the mounting projection 88 causes rotation of the take-up roll 74 engaging therewith. Rotation of the take-up roll 74 exerts a pulling action on the lining materials 26, 62 of the upper and lower rolls 24, 64 causing the waste to be transported by the lining material 62 of the lower roll 64 over the lower shelf 32 to the end of the slot 20. At the end of the slot 20, the waste falls through opening 42 into a space defined between the lining materials 26, 62 of the upper and lower rolls 24, 64 (see the waste in FIG. 3).

Continued rotation of the take-up roll 74, either during the same downward pulling of the handle 78 or subsequent downward pulling(s) of the handle 78, causes the waste to be enclosed between the lining materials 26, 62 of the upper and lower rolls 24, 64 and the formation of a roll 60 of enclosed waste and lining material (see FIGS. 1 and 3). Additional waste can be placed into the slot 20 and added to the roll 60 until the roll 60 substantially fills the lower compartment 28. This might be indicated by resistance to the downward pulling of the handle 78.

To remove the roll 60, the door 54 to the lower compartment 28 is opened thereby releasing the locking projection 164 of the lock-out arm 160 from engagement with the annular groove 156 of the main body part 148 of the release mechanism 98. The roll 60 of enclosed waste and lining material is then held while the knob 152 is pulled outward releasing the projection 96 from engagement with the take-up roll 74 (see FIG. 9). The roll 60 of enclosed waste and lining material is then pulled downward at an angle to release the take-up roll 74 from engagement with the mounting projection 88. The roll 60 of enclosed waste and lining material is then pulled downward in the direction of arrow B so that a length of lining material 26, 62 from both the upper and lower rolls 24, 64 is present in the lower compartment 28 without any waste therebetween. Then, the lining material 26, 62 from both the upper and lower rolls 24, 64 is torn along perforation lines 146 while maintaining a length of the lining materials 26, 62 in the lower compartment 28. This length of lining material 26, 62 from both the upper and lower rolls 24, 64 is adhered to the adhesive material 84 on a new take-up roll 74, after a protective coating layer on the adhesive material 84 is peeled off. The new take-up roll 74 is positioned between the mounting projection 88 and the projection 96 on the release mechanism 98 in the same manner as described above (see FIG. 10).

Referring now to FIGS. 12-16, alternative attachment mechanism 82 which enable the lining materials 26, 62 of the upper and lower rolls 24, 64 to be attached to the take-up roll 74 are shown. To the extent not inconsistent, the description above relating to the installation, use, removal and replacement of the take-up roll 74 with the adhesive attachment mechanism applies to the attachment mechanisms described below.

In FIG. 12, the take-up roll 74 includes an axially extending slot 176 having a first end proximate the end 74b of the take-up roll 74 which is mounted to the projection 96 of the release mechanism 98 and a second end proximate the end 74a of the take-up roll 74 mounted onto the projection 88. The slot 176 extends entirely through the interior of the take-up roll 74 so that the lining materials 26, 62 of the upper and lower rolls 24, 64 can be passed through the slot 176 and draped over the take-up roll 74 as shown in FIG. 12. Upon rotation of the take-up roll 74, the lining materials 26, 62 will be entrained with the take-up roll 74 and pulled downward. The take-up roll 74 may be manually rotated prior to mounting of the take-up roll 74 in the lower compartment 28 to ensure that the lining materials 26, 62 are entrained with the take-up roll 74. In the alternative, the take-up roll 74 with the lining materials 26, 62 draped over it can be mounted and then the take-up roll 74 rotated by pulling the handle 78 downward.

In FIG. 13, the take-up roll 74 includes barbs 178 formed on an outer surface and having a sharp forward edge, forward in the sense that the sharp edge will first contact the lining materials 26, 62 of the upper and lower rolls 24, 64 upon rotation of the take-up roll 74. As such, attachment of the lining materials 26, 62 of the upper and lower rolls 24, 64 to the take-up roll 74 entails rotating the take-up roll 74 to cause the barbs 178 to pierce the lining materials 26, 62 of the upper and lower rolls 24, 64 and thereby entrain the lining materials 26, 62 with the take-up roll 74. Once entrained, the door 54 to the lower compartment 28 is closed and the device 10 is ready for use. Upon further operational rotation of the take-up roll 74, the lining materials 26, 62 will be pulled downward. FIG. 13 also shows the possibility of tapering the axial end 74a of the take-up roll 74.

In FIG. 14, the take-up roll 74 comprises a disposable cardboard take-up spool 180 and reusable inserts 182,184, one at each end of the take-up spool 180. The take-up spool 180 includes discrete adhesive bands 186, optionally and preferably covered by removable peel strips (not shown), to which the forward edge portions of the lining materials 26, 62 of the upper and lower rolls 24, 64 are adhered. The take-up roll 74 also includes axially extending slots 188 formed at one end.

A first insert 182 is designed to secure the take-up spool 180 to the mounting projection 88 to enable the transmission of rotational force from the mounting projection to the take-up spool 180. To this end, the first insert 182 includes axially extending slots 190 at one end adapted to mate with the ribs 92 on the mounting projection 88 and axially extending tabs 192 at the opposite end adapted to mate with the axially extending slots 188 formed at the end of the take-up spool 180.

A second insert 184 is designed to secure the take-up spool 180 to the projection 96 while enabling the take-up spool 180 to rotate relative to the projection 96. To this end, the second insert 184 includes a cylindrical portion 194 insertable into the axial end of the take-up spool 180 and a tubular portion 196 adapted to receive the projection 96.

Removal of the take-up roll 74 shown in FIG. 14 from its mounting in the lower compartment is as discussed above. However, instead of throwing out the entire take-up roll 74 with the enclosed waste and lining material rolled thereon, the inserts 182, 184 are separated from the take-up spool 180 and only the take-up spool 180 and enclosed waste and lining material rolled thereon are discarded. Instead of an entirely new take-up roll, only a new take-up spool 180 is needed and is attached to the inserts 182, 184 used for the previous take-up spool 180.

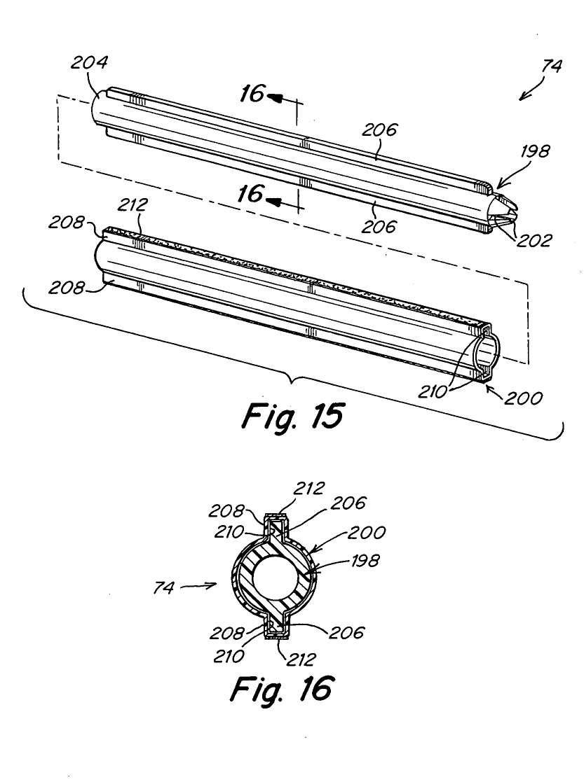

In the embodiment shown in FIGS. 15 and 16, the take-up roll 74 comprises a reusable, substantially tubular core 198 and a disposable plastic sleeve 200. The core 198 includes axially extending slots 202 at one axial end adapted to mate with the ribs 92 on the mounting projection 88 and a tubular portion 204 at the other axial end adapted to receive the projection 96 of the release mechanism 98. The core 198 also includes ridges 206 which may be diametrically opposed or evenly spaced around the circumference of the core 198. This enables the construction and use of disposable sleeves 200 having ridges 208 defining inner grooves 210 in which the ridges 206 of the core 198 are received (see FIG. 16). Adhesive strips 212 are provided on the outer, radial surface of the ridges 208 and optionally covered by a peel strip (not shown).

To mount the take-up roll 74 in the lower compartment 28, a sleeve 200 is placed on a core 198 and then the core 198 is engaged with the projection 96 on the release mechanism 98 and the mounting projection 88 (as described above). To adhere the lining materials 26, 62 to the take-up roll 74, the peel strips are removed from the adhesive strips 212 and the lining material 26 of the upper roll 24 is adhered to one adhesive strip 212 and the lining material 64 of the lower roll 62 is adhered to another adhesive strip 212. Adhesion of the lining materials 26, 62 to the take-up roll 74 may be performed before or after the take-up roll 74 is mounted in the lower compartment 28.

Removal of the roll 60 of enclosed waste and lining material would entail dismounting the take-up roll 74 from its position in the lower compartment 28 (in the manner described above) and the sliding the sleeve 200 off of the core 198. The roll 60 of enclosed waste and lining material is formed in connection with the sleeve 200 and not the core 198 so only the sleeve 200 has to be discarded and cannot be reused. A new sleeve 200 is then placed on the core 198 and the take-up roll 74 thus-formed is remounted in the lower compartment 28.

A second embodiment of a waste disposal system 300 in accordance with the invention is shown in FIGS. 17-20 and comprises a housing 312 having a front wall 314 having a curved top portion 314a and a curved bottom portion 314b, a rear wall 316 having a curved top portion 316a and a curved bottom portion 316b and opposed side walls 318. Housing 312 includes a horizontal slot or chute 320 into which waste products are placed. An upper compartment 322 is formed above the slot 320 and receives a roll 324 of lining material 326 and a lower waste-receiving compartment 328 is formed below the slot 320.

Slot 320 is defined by an upper shelf 330, a lower shelf 332 and opposed side walls 334. The upper shelf 330 extends forward and upward from the rear wall 316 and supports the roll 324 of lining material 326. The lining material 326 unwinds from the roll 324 through a dispensing slot 338 defined between a lip 308 of the upper shelf 330 and a lower edge 306 of a door 344 providing access to the upper compartment 322. The door 344 is pivotally connected to the curved top portion 316a of the rear wall 316 by hinges 346 and extends only partly across the length of the front wall 314. As such, the housing 312 includes interior walls 304 constituting the side walls of the upper compartment 322. The door 344 is biased closed by spring action. Access to the upper compartment 322 is necessary in order to insert replacement rolls 324 of lining material 326.

The lower shelf 332 extends inward and downward from the front wall 314 of the housing 312. A dispensing compartment 310 is formed in the housing 312 below the lower shelf 332 and receives a roll 364 of lining material 362. The dispensing compartment 310 has a rear wall 366, a lower wall 368 and side walls 370. The upper wall of the dispensing compartment 310 is the lower shelf 332. The dispensing compartment 310 is accessible by means of a door 372 pivotally connected to the front wall 314. A dispensing slot 374 is formed between the door 372 and the lower shelf 332. The door 372 includes a catch 376 on each side engaging with the front wall 314. Pressing the catches 376 outward (or inward depending on the construction of the catches 376) releases the door 372 from engagement with the front wall 314 and causes the door 372 to pivot about one or more hinges and exposes the dispensing compartment 310. Access to the dispensing compartment 310 is necessary in order to insert new rolls 364 of lining material 362.

The lower compartment 328 is accessible by means a door 352 pivotally connected to the curved bottom portion 316b of the rear wall 316 (or to the curved bottom portion 314b of the front wall 314) by hinges 354. The door 352 includes a resilient latch or clasp 356 engaging with the front wall 314. Pressing the latch 356 inward against its bias releases a shoulder 358 from engagement with the inner surface of the front wall 314 and then pulling the latch 356 away from the front wall 314 causes the door 352 to pivot about the hinges 354 and exposes the lower compartment 328. Access to the lower compartment 328 is necessary in order to remove the roll 416 of waste enclosed by the lining materials 326, 362.

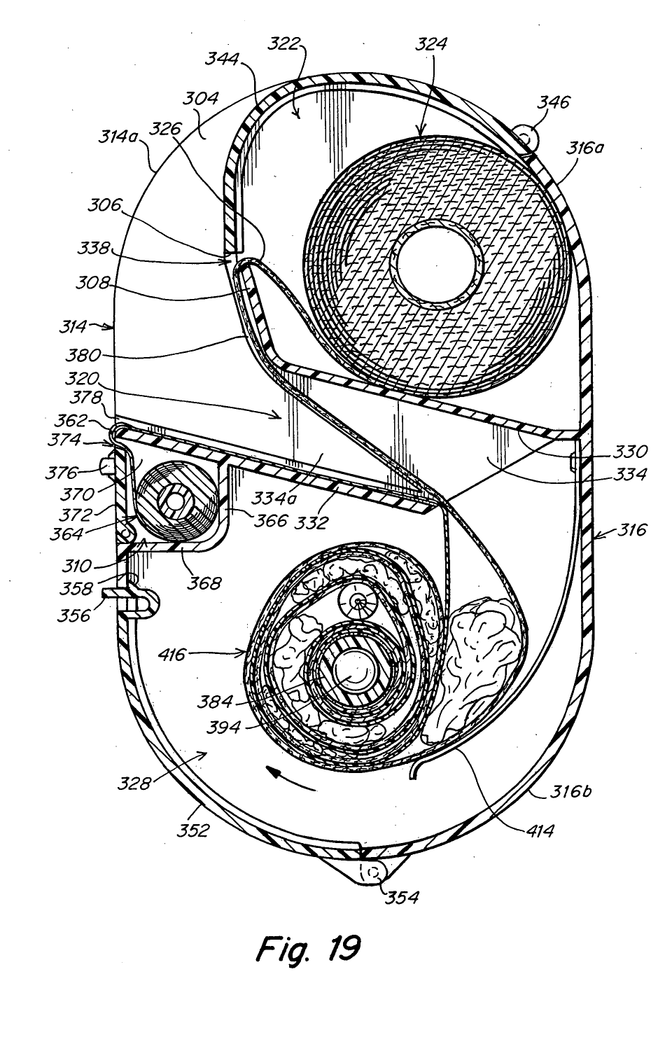

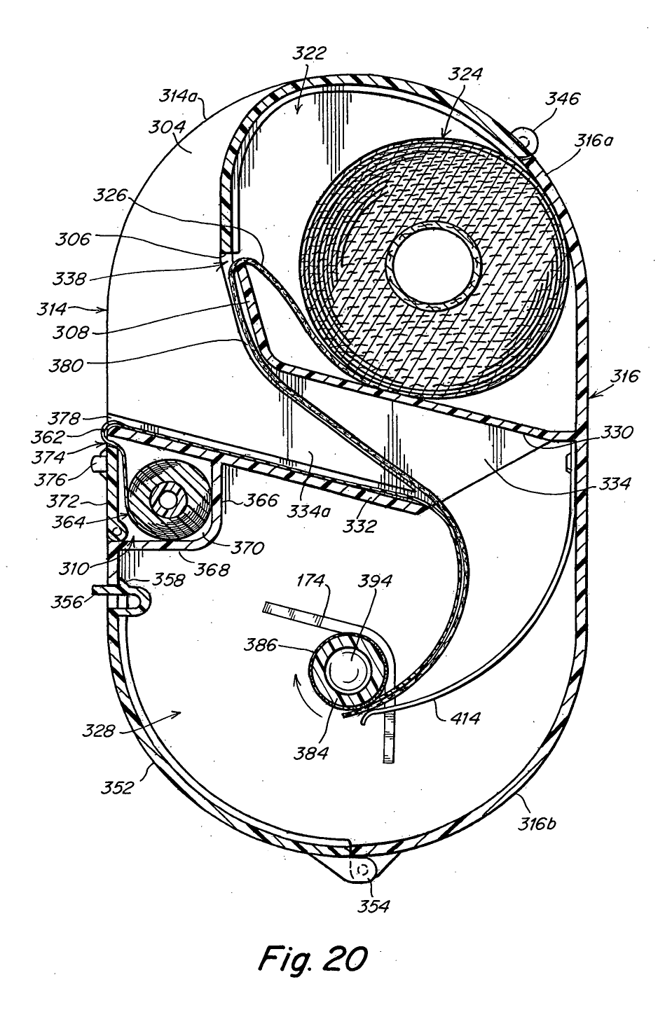

In this embodiment, the waste is enclosed between lining material 326 from the upper roll 324 in the upper compartment 322 and lining material 362 from the lower roll 364 in the dispensing compartment 310 to form a roll 416 of enclosed waste and lining material in the lower compartment 328. The lower roll 364 of lining material 362 unwinds in a clockwise direction with the lining material 362 passing through the dispensing slot 374 and in a slot 378 defined by the housing 310 above the lower shelf 332 (see FIGS. 19 and 20). The upper roll 324 of lining material 326 unwinds in a clockwise direction with the lining material 326 passing through the dispensing slot 338 and in a slot 380 defined by the housing 312 below the upper shelf 330 (see FIGS. 19 and 20). Since the lining material 362 of the lower roll 364 passes over the upper surface of the lower shelf 332 and the lining material 326 of the upper roll 324 passes below the lower surface of the upper shelf 330, the upper and lower shelves 330, 332 are not exposed to contaminants from the waste and thus shelf protectors for either or both of these shelves are not required.

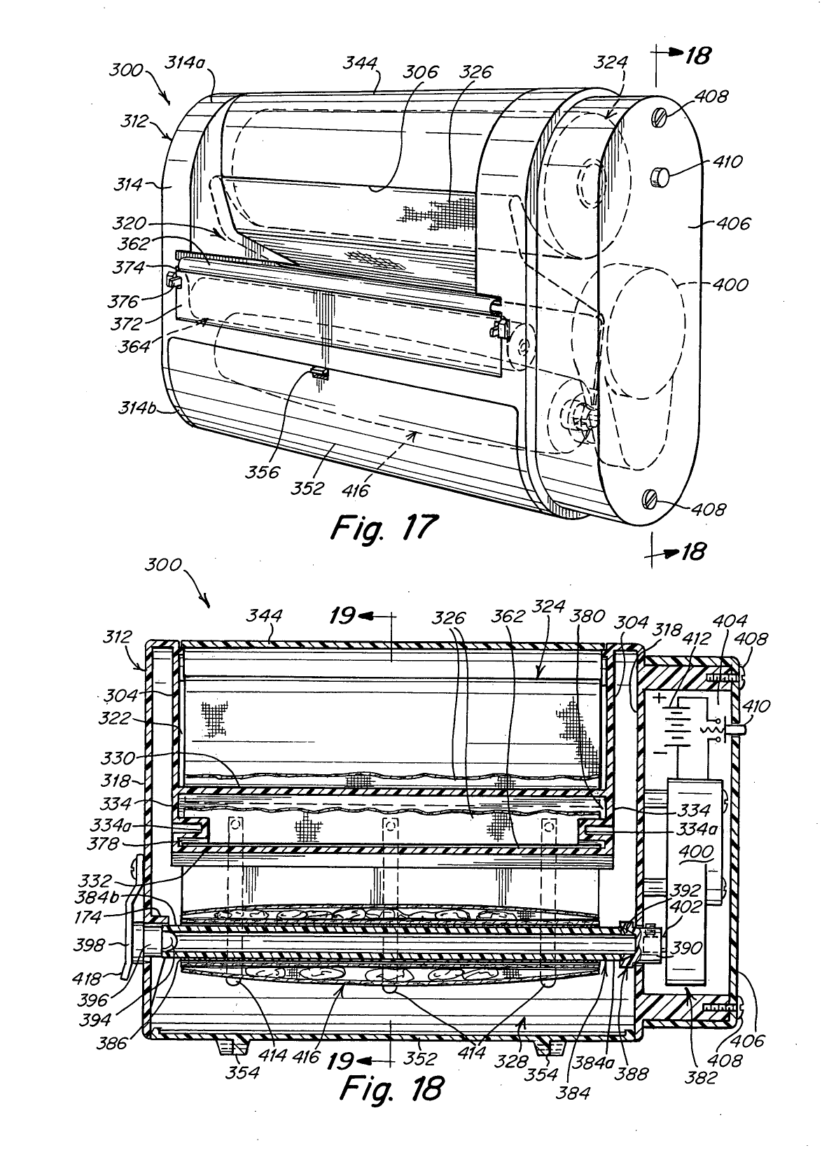

The width of the lining material 362 in the lower roll 364 may be substantially the same as the width of the lining material 326 in the upper roll 324. However, to prevent a situation where waste at an edge of the lining material 326, 362 is not entrained, the width of the slot 320 is shortened to be less than that of the lining material 326, 362 of the upper and lower rolls 324, 364. This may be achieved by providing inwardly directed projections 334a in the lateral walls 334 of the housing 312 defining the slot 320 (see FIG. 18). Thus, any waste placed into the slot 320 is not situated at an edge of the lining materials 326, 362 and the edges of the lining material 326, 362 from the upper and lower rolls 324, 364 come together to enclose the waste therebetween.

The slot 380 for the lining material 326 from the upper roll 324 guides the lining material 326 downward to a lower edge of the slot 320, at which the lining material 326 from the upper roll 324 will contact the lining material 362 from the lower roll 364 in the absence of waste therebetween. When waste is situated in the slot 320, the lining material 326 from the upper roll 324 can rise upward in the slot 320.

A mechanism for forming a roll of enclosed waste and lining material in this embodiment comprises a take-up roll 384 rotatably mounted in the lower compartment 328 and a rotation mechanism 382 for rotating the take-up roll 384.

The take-up roll 384 includes an attachment mechanism 386 to enable the lining material of the upper and lower rolls to be attached thereto. The attachment mechanism 386 may be adhesive arranged on the cylindrical outer surface of the take-up roll 384. Other take-up rolls with various attachment mechanisms which might be used in this embodiment are described above with reference to FIGS. 12-16.

The take-up roll 384 is tubular and at one end is mounted onto a bayonet-type mounting projection portion 388 of a coupling 390 passing through a side wall 318 of the housing 312 (see FIG. 18). To this end, the end 384a of the take-up roll 384 includes axially extending slots which are designed to receive axially extending ribs 392 formed on the mounting projection portion 388. The ribs 392 are formed around the periphery of the mounting projection portion 388 and thus, the mounting projection portion 388 does not pass into the interior of the take-up roll 384 (in contrast to the bayonet-type mounting projection described above with reference to FIG. 5). Instead of a ribbed mounting projection and corresponding axially extending slots formed in the take-up roll 384, the reverse situation can be provided (ribs on the take-up roll 384 and slots in the mounting projection portion 388) and other mechanisms to providing rotational engagement between the coupling 390 and the take-up roll 384 can be applied in the invention.

At the other axial end 384b, the take-up roll 384 is mounted onto a projection 394 formed on a biasing member 396. The biasing member 396 is biased to maintain the projection 394 in engagement with the take-up roll 384 by a spring arm 398 attached to the side wall 318 of the housing 312 (see FIG. 18). A lock-out arm can be arranged in connection with the biasing member 396, i.e., by providing an annular groove on the biasing member 396 and mounting the lock-out arm in the same manner as described above. In this case, opening of the door 352 to the lower compartment 328 would be required to remove the take-up roll 384.

Instead of the biasing member 396 and spring arm 398, other mechanisms for maintaining the projection 394 in engagement with the take-up roll 384 can also be provided, including the release mechanism 98 described above. Similarly, the biasing member 396 and spring arm 398 can be used in the embodiment described above instead of the release mechanism 98.

The rotation mechanism 382 comprises an electric motor 400 arranged to rotate an output shaft 402. The motor 400 and output shaft 402 are arranged in a compartment 404 alongside the housing 312 defined between the side wall 318 and a cover plate 406 attached to the side wall 318 by an appropriate attachment mechanism, such as screws 408 and the like. A push-button switch 410 to actuate the motor 400 extends through the cover plate 404 to the side thereof. A battery 412 for powering the motor 400 is also provided in the compartment 404 and a door (not shown) to enable replacement of the battery 412 can be formed in the cover plate 404. The motor 400 may be any conventional motor including for example, a timer and a gear reduction unit.

The output shaft 402 is connected to the coupling 390 so that rotation of the output shaft 402, caused by depression of the switch 410, translates into rotation of the coupling 390 and thus rotation of the take-up roll 384. Other mechanisms for converting a manual motion into actuation of a motor to cause the motor to rotate the take-up roll 384 can also be applied in the invention.

A mechanism may be provided to facilitate retention of waste being rolled onto the take-up roll 384 to form the roll 416 of enclosed waste and lining material. For example, such a mechanism may comprise spring fingers 414 attached to the rear wall 318 of the housing 312 and biased to press the lining material 326, 362 from the upper and lower rolls 324, 364 against the take-up roll 384. In the illustrated embodiment, there are three fingers 414, one in the approximate axial middle of the take-up roll 384 and one proximate each axial end 384a, 384b of the take-up roll 384. The pressure exerted by the fingers 414 at the axial ends 384a, 384b of the take-up roll 384 facilitates the retention of waste in the roll 416 by preventing the passage of waste from between the lining materials 326, 362 of the upper and lower rolls 324, 364 axially outward. Although three fingers 414 are shown, any number of fingers 414 can be used in the invention.

The fingers 414 serve several purposes. First, upon placing new upper and lower rolls 324, 364 in the waste disposal system 300, the lining materials 326, 362 from the upper and lower rolls 324, 364 can be automatically engaged with the take-up roll 384, provided the take-up roll 384 has an exposed adhesive portion 386. More particularly, the forward edge portions of the lining materials 326, 362 of the upper and lower rolls 324, 364 is pushed down the slot 320 until they buckle slightly indicating that the forward edges are sandwiched at a nip between the fingers 414 and the take-up roll 384. The fingers 414 thus press the forward edge portions against the adhesive 386 of the take-up roll 384.

Second, in the take-up roll replacement procedure, the lining materials 326, 362 from both the upper and lower rolls 324, 364 is torn along perforation lines. After the take-up roll 384 is replaced and an adhesive portion 386 exposed, the forward edge portion of the remaining length of lining materials 326, 362 is pressed by the fingers 414 against the exposed adhesive portion 386 of the take-up roll 384 (see FIG. 20). In this manner, a continuous attachment mechanism is provided for sequentially attaching the lining materials 326, 362 to a plurality of take-up rolls 384.

Third, the constant pressing of the fingers 414 in the direction of the take-up roll 384 compacts the waste between the lining materials 326, 362 and prevents sagging of the lining material 326 of the upper roll 324 when heavy waste is placed in the slot 320 and drops out of the slot 320 (see FIG. 19).

The features in the embodiments described above with reference to FIGS. 1-16 can also be applied in the embodiment of FIGS. 17-20 and vice versa, to the extent possible. For example, the embodiment of FIGS. 17-20 can include the same mounting mechanism 140 for mounting the housing 312 to a wall or other vertical surface, the same lining materials, and the same guide ramps 172 and locating rim 174.

To prepare the waste disposal system 300 for use, the housing 312 is mounted to a wall and the doors 344, 352 are opened and rolls 324, 364 of lining material 326, 362 are prepared. An upper roll 324 is placed into the upper compartment 322 and unwound slightly until a forward edge portion extends beyond the upper lip 308 of the upper shelf 330 and then the door 344 is closed to thereby cause the lining material 326 to pass through the dispensing slot 338. A lower roll 364 is placed into the dispensing compartment 310 and unwound slightly. The forward edge portion is grasped as the door 372 is closed to thereby cause the lining material 364 to pass through the dispensing slot 374.

A take-up roll 384 is mounted in the housing 312, either before, during or after the upper and lower rolls 324, 364 are placed into the upper and lower dispensing compartments 322, 310, respectively. Mounting of the take-up roll 384 involves opening the door 352 of the lower compartment 328, forcing the living spring hinge arm 398 outward away from the side wall 318 (by applying outward pressure to the lower tab 418 of the spring arm 398), placing the end 384a of the take-up roll 384 with the axially extending slots on the mounting projection portion 388 of the coupling 390, then guiding the other axial end 384b of the take-up roll 384 along the locating rim 174 until it is in alignment with the projection 394 on the biasing member 396 and finally releasing the force applied to the spring arm 398 to cause the biasing member 396 to move inward and the projection 394 thereon to pass into a space at the axial end 384b of the take-up roll 384. A peel strip covering the adhesive 386 on the take-up roll 384 is removed and then the door 352 of the lower compartment 328 is closed.

After the take-up roll 384 is mounted in the housing 312, the lining material 326, 362 from the upper and lower rolls 324, 364 is forced through the respective slot 378, 380 into the lower compartment 328 until they buckle slightly indicating that the forward edges are sandwiched at a nip between the fingers 414 and the take-up roll 384. The fingers 414 press the forward edge portions of the lining materials 326, 362 against the adhesive 386 of the take-up roll 384 and the waste disposal system 300 is ready for use (in the position shown in FIG. 20).

In one exemplifying use, waste is placed into the slot 320 onto the lining material 362 from the lower roll 364. In view of the downward inclination of the lower shelf 332, the waste does not fall forward out of the slot 320. The switch 410 is then depressed to cause the motor 400 to rotate the output shaft 402, leading to rotation of the coupling 390 and thus rotation of the take-up roll 384. Rotation of the take-up roll 384 exerts a pulling action on the lining material 326, 362 of the upper and lower rolls 324, 364 causing the waste to be transported by the lining material 362 of the lower roll 364 over the lower shelf 332 to the end of the slot 320 and into engagement with the lining material 326 from the upper roll 324.

Continued rotation of the take-up roll 384, either during the same depression of the switch 410 or subsequent depression(s) of the switch 410, causes the waste to be enclosed between the lining materials 326, 362 of the upper and lower rolls 324, 364 and the formation of a roll 416 of enclosed waste and lining material (see FIG. 19). During the formation of the roll 416 of enclosed waste and lining material, the fingers 414 flex outward before waste passes through the nip defined between the fingers 414 and the roll 416 of waste and lining material. In the nip, the fingers 414 exert pressure against the waste to cause the waste to be compacted. After the waste passes through the nip, the fingers 414 flex inward. The flexing of the fingers 414 is designed to create a compact roll 416 of enclosed waste and lining material. Additional waste can be placed into the slot 320 and added to the roll 416 until the roll 416 substantially fills the lower compartment 328.

To remove the roll 416, the door 352 to the lower compartment 328 is opened by pressing the latch 356 downward and pulling it outward. The roll 416 of enclosed waste and lining material is then held while the spring arm 398 is pulled outward releasing the projection 394 from engagement with the take-up roll 384. The roll 416 of enclosed waste and lining material is then pulled downward at an angle to release the take-up roll 384 from engagement with the mounting projection portion 388 of the coupling 390. The roll 416 of enclosed waste and lining material is then pulled downward so that a length of lining material 326, 362 from both the upper and lower rolls 324, 364 is present in the lower compartment 328 without any waste therebetween. Then, the lining material 326, 362 from both the upper and lower rolls 324, 364 is cut or torn along perforation lines while maintaining a length of the lining material 326, 362 in the lower compartment 328. This length of lining material 326, 362 from both the upper and lower rolls 324, 364 is adhered to the adhesive material 386 on a new take-up roll 384, after a protective coating layer on the adhesive material 386 is peeled off and the new take-up roll 384 is positioned between the mounting projection portion 388 of the coupling 390 and the projection 394 on the biasing member 396 in the same manner as described above for the initial installation of a take-up roll 384. The fingers 414 press the torn ends of the lining material 326, 362 against the adhesive 386 and after the door 352 to the lower compartment 322 is closed, the system 300 is again ready for use.

In the embodiments of the invention described above with reference to FIGS. 1-20, two rolls 324, 364 of lining material 326, 362 are used with waste being sandwiched between the lining material 326 from one roll 324 and the lining material 362 from the other roll 364. Alternatively, it is possible to use a single roll of lining material with the waste being sandwiched between successive windings of the lining material about the take-up roll.

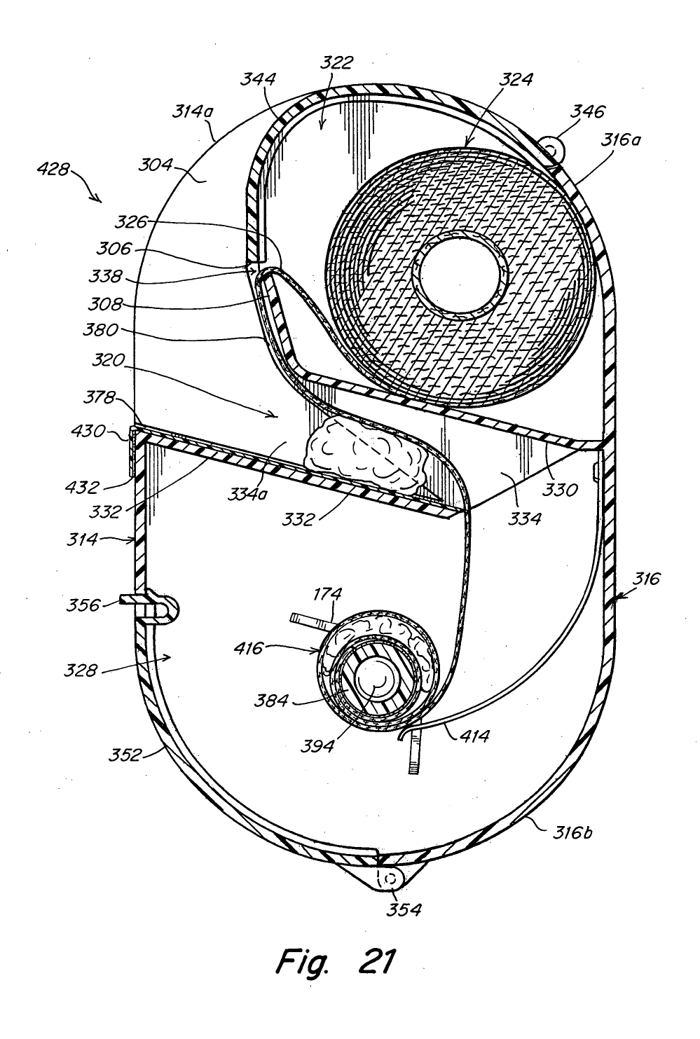

To this end, FIG. 21 shows a waste disposal system 428 which is similar to the one shown in FIGS. 17-20 with the exception that a lower dispensing compartment for housing a lower roll of lining material is not provided.

In view of the absence of a lower roll of lining material, and since it is beneficial to prevent the accumulation of waste on the lower shelf 332, a substantially L-shaped shelf protector 430 is arranged partially on the outer surface of the front wall 314 (the short segment of the L) and partially on the upper surface of the lower shelf 332 (the long segment of the L) to prevent the accumulation of waste particles on the upper surface of the lower shelf 332. Preferably, the shelf protector 430 is disposable so that it can be replaced after a set period of time, e.g., once a day. To this end, the shelf protector 430 includes a mechanism for detachably mounting it to the housing, specifically, the outer surface of the front wall 314 and/or the upper surface of the lower shelf 332. The replaceable shelf protectors 430 could thus include adhesive 432 on a rear surface of the short segment of the L with a layer of coating material which is peeled off of the adhesive upon placement of the shelf protector 430 in connection with the lower shelf 332.

The lining material 326 in the upper roll 324 may be designed to have a paper fabric side and a plastic coating side with the paper fabric side coming into contact with the waste in the slot 320.

To prepare the waste disposal system 428 for use, the door 344 to the upper dispensing compartment 322 is opened and a roll 324 of lining material 326 is placed therein and unwound slightly until a forward edge portion extends beyond the upper lip 308 of the upper shelf 330 and then the door 344 is closed to thereby cause the lining material 326 to pass through the dispensing slot 338. A shelf protector 430 is placed over the lower shelf 332 by removing the coating material to expose the adhesive 432 and pressing the adhesive 432 against the outer surface of the front wall 314.

A take-up roll 384 is mounted in the housing 312 in the same manner as discussed above with reference to FIGS. 17-20. After the take-up roll 384 is mounted in the housing 312, the lining material 326 from the upper roll 324 is forced through the slot 320 into the lower compartment 328 until it buckles slightly indicating that the forward edge is sandwiched at a nip between the fingers 414 and the take-up roll 384. The fingers 414 press the forward edge portion of the lining material 326 against the adhesive 386 of the take-up roll 384 and the waste disposal system 428 is ready for use.

In one exemplifying use, waste is placed into the slot 320 onto the shelf protector 430 and forward enough to be in engagement with the lining material 326 from the upper roll 324. The switch 410 is depressed to cause the motor 400 to rotate the output shaft 402, leading to rotation of the coupling 390 and thus rotation of the take-up roll 384. Rotation of the take-up roll 384 exerts a pulling action on the lining material 326 of the upper roll 324 causing the waste to be transported by the lining material 326 of the upper roll 324, via the frictional engagement of the paper fabric side of the lining material 326 and the waste, to the end of the slot 320. Friction inducing agents could also be included in the lining material 326 or on the side of the lining material 326 facing the waste.

Continued rotation of the take-up roll 384 causes the waste to fall out of the slot 320 toward the nip between the fingers 414 and the take-up roll 384. The fingers 414 press the waste against a winding of lining material 326 previously wound onto the take-up roll 384. As such, the waste is enclosed between successive windings of the lining material 326. A full roll of enclosed waste and lining material is removed in the same manner as in the embodiment of FIGS. 17-20.

Instead of the motorized rotation mechanism shown in FIGS. 17-20, the embodiment shown in FIG. 21 can also use the rotation mechanism 76 shown in FIGS. 1-10.

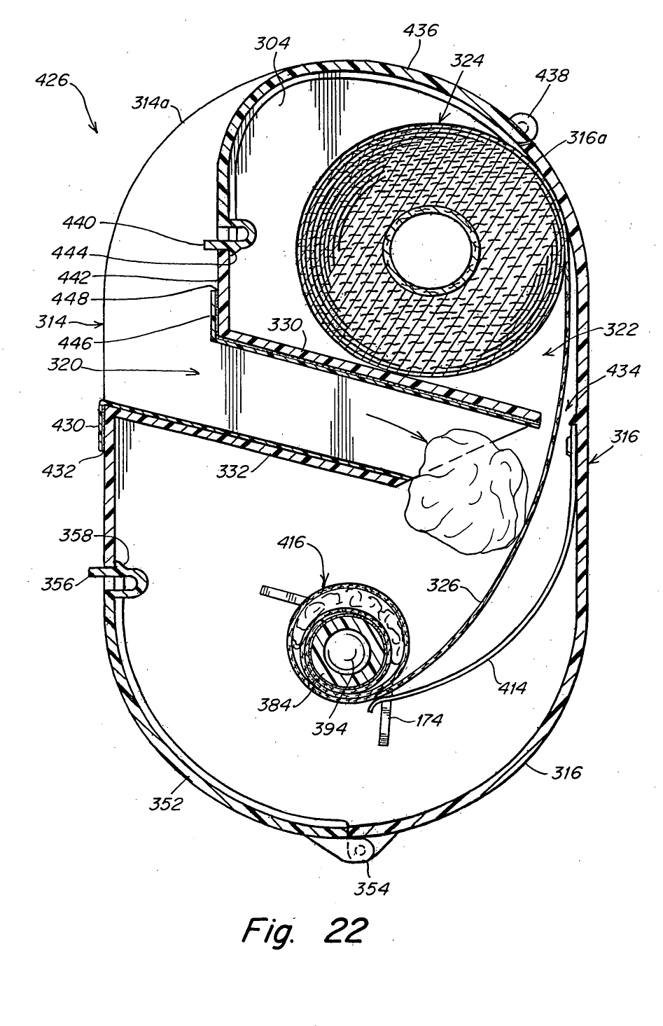

FIG. 22 shows an embodiment of a waste disposal system 426 which differs from the embodiment shown in FIG. 21 in that a dispensing slot 434 for the upper roll 324 of lining material 326 is situated at a rear of the housing 312 between a rear edge of the upper shelf 330 and the rear wall 316 of the housing 312 so that the lining material 326 does not pass through the slot 320. The upper shelf 330 may be connected to the side walls 304 defining the upper compartment 322. Also, a slot 380 for the lining material 326 from the upper roll 324 is not provided as the lining material 326 does not pass through the slot 320.

The upper compartment 322 is accessible by means of a door or flap 436 pivotally connected to the curved top portion 316a of the rear wall 316 by hinges 438. The door 436 includes a resilient latch or clasp 440 engaging with a front intermediate wall 442 of the housing 312 which is connected to the upper shelf 330. Pressing the latch 440 inward against its bias releases a shoulder 444 from engagement with the inner surface of the front wall 442 and then pulling the latch 440 away from the front wall 442 causes the door 436 to pivot about the hinges 438 and exposes the upper compartment 322. Other mechanisms for mounting and securing the door 436 to the housing 312 are also envisioned and within the scope of the invention.

A second, substantially L-shaped shelf protector 446 is optionally arranged partially on the outer surface of the front wall 442 (the short segment of the L) and partially on the lower surface of the upper shelf 330 (the long segment of the L) to prevent the accumulation of waste particles on the lower surface of the upper shelf 330. Preferably, the shelf protector 446 is disposable so that it can be replaced after a set period of time. The shelf protector 446 includes a mechanism for detachably mounting it to the housing 312, specifically, the outer surface of the front wall 442 and/or the lower surface of the upper shelf 330. The replaceable shelf protectors 446 could thus include adhesive 448 on a rear surface of the short segment of the L and on a rear surface of the long segment of the L with a layer of coating material which is peeled off of the adhesive upon placement of the shelf protector in connection with the front wall 442 and upper shelf 330.

To prepare the waste disposal system 426 for use, a take-up roll 384 is mounted in the housing 312 in the same manner as discussed above with reference to FIGS. 17-20. The door 436 to the upper dispensing compartment 322 is opened and a roll 324 of lining material 326 is placed therein. The lining material 326 is forced through the slot 434 into the lower compartment 328 until it buckles slightly indicating that the forward edge is sandwiched at a nip between the fingers 414 and the take-up roll 384. The fingers 414 press the forward edge portion of the lining material 326 against the adhesive 386 of the take-up roll 384. The door 436 to the upper compartment 322 is closed. A shelf protector 430, 446 is placed over the lower and/or upper shelf 330, 332 by removing the coating material to expose the adhesive 432, 448 and pressing the adhesive 432, 448 against the respective surfaces of the housing 312. The waste disposal system 426 is ready for use.

In one exemplifying use, waste is placed into the slot 320 onto the shelf protector 430 and pushed to the end of the slot 320 to fall onto the lining material 326 from the upper roll 324. The switch 410 is depressed to cause the motor 400 to rotate the output shaft 402, leading to rotation of the coupling 390 and thus rotation of the take-up roll 384. Rotation of the take-up roll 384 exerts a pulling action on the lining material 326 of the upper roll 324 causing the waste to be urged toward the nip between the fingers 414 and the take-up roll 384, via frictional engagement between the fabric paper side of the lining material 326 and the waste. The fingers 414 press the waste against a winding of lining material 326 previously wound onto the take-up roll 384. As such, the waste is enclosed between successive windings of the lining material 326. A full roll of enclosed waste and lining material is removed in the same manner as in the embodiment of FIGS. 17-20.

Instead of the motorized rotation mechanism shown in FIGS. 17-20, the embodiment shown in FIG. 22 can also use the rotation mechanism 76 shown in FIGS. 1-10.

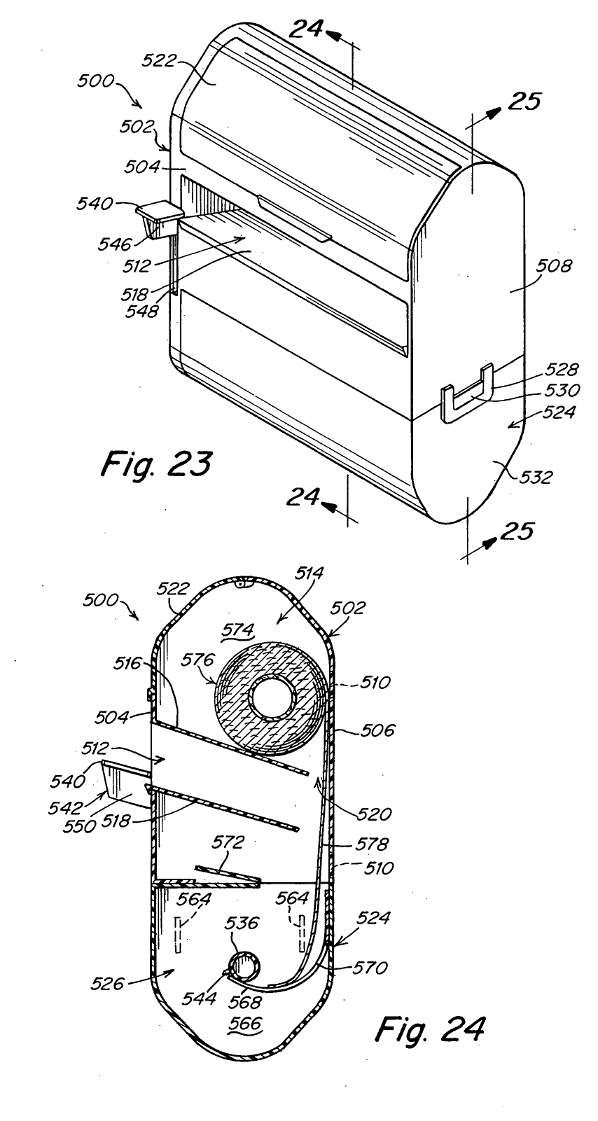

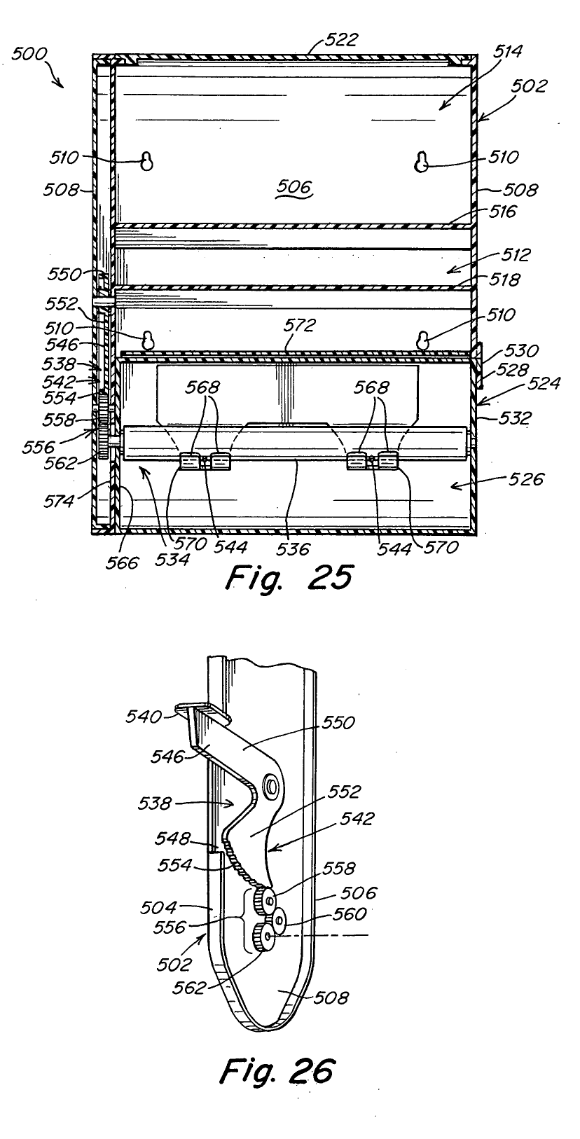

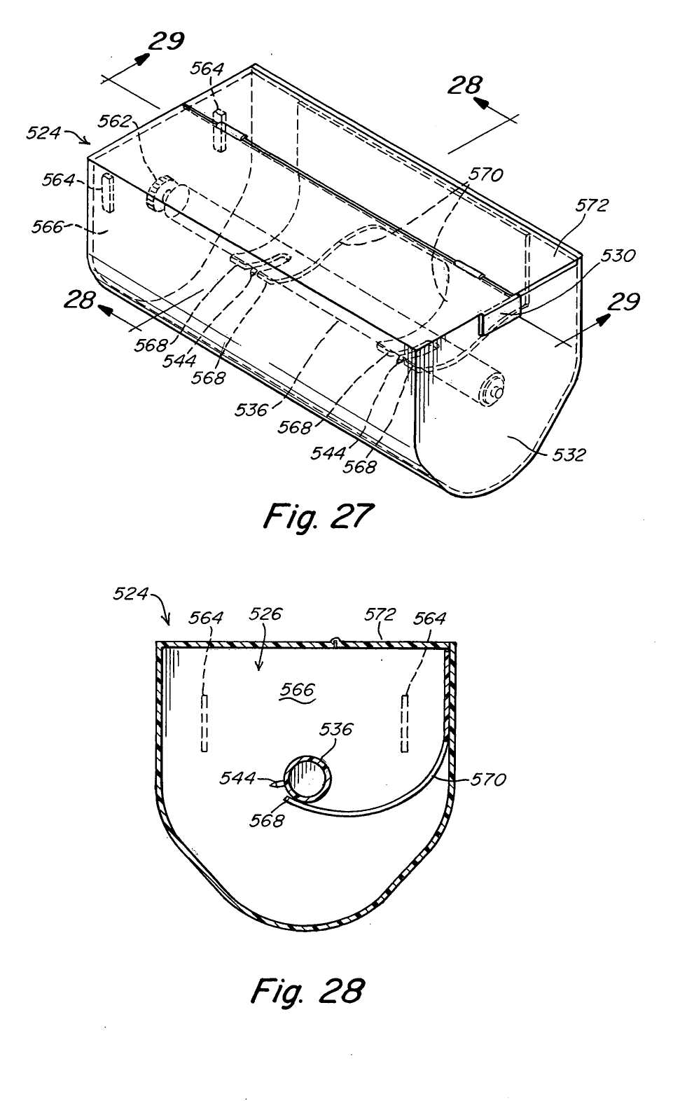

FIGS. 23-29 show another embodiment of a waste disposal system 500 in accordance with the invention which uses a single roll of lining material with the waste being sandwiched between successive windings of the lining material about the take-up roll. The waste disposal system 500 generally comprises a housing 502 having a front wall 504, a rear wall 506 connected at its top and bottom to the top and bottom of the front wall 504, respectively, and opposed side walls 508. Mounting mechanisms may be provided on or in connection with the rear wall 506 for mounting the housing 502 to a wall or other vertical surface, e.g., apertures 510 formed in the rear wall 506 as shown in FIG. 24 or the mounting mechanisms described above such as suction cups.

Housing 502 includes a horizontal chute or slot 512 into which waste products are placed. A dispensing compartment 514 which receives a roll of lining material 576 is formed above the slot 512. Slot 512 can extend only partially or fully across the length of the housing 502 between the opposed walls 508.

Slot 512 is substantially rectangular and is defined by an upper shelf 516 and a lower shelf 518. Optionally, opposed lateral walls may be provided, for example, when the slot 512 does not extend entirely between the opposed walls 508. The upper shelf 516 extends inward into the interior of the housing 502 and downward. The upper shelf 516 supports the roll of lining material 576 and the rear end of the upper shelf 516 is close enough to the rear wall 506 to prevent the roll of lining material 576 from falling, even when the lining material 578 is exhausted from the core of the roll 576. The lining material 578 passes through a dispensing slot 520 formed between the rear edge of the upper shelf 516 and the rear wall 506 to be situated inward of the inward end of the slot 510.

The lower shelf 518 also extends inward into the interior of the housing 502 and downward. The upper and lower shelves 516, 518 may be parallel to one another or otherwise, e.g., angling toward one another at the inward ends. Optionally, the inward end of the slot 512 may be provided with an angled opening as shown in FIG. 3. Also, shelf protectors as described above may be used to cover one or both shelves 516, 518.

The upper compartment 514 is accessible by means of a door or flap 522 pivotally connected to a curved top portion of the front or rear wall 504, 506. Access to the upper compartment 514 is necessary in order to insert replacement rolls of lining material 576. The door 522 may be mounted and secured to the housing 502 in the same manner as in the embodiments described above.

The housing 502 includes a detachable bottom section 524 which defines a waste-receiving compartment 526. To secure the bottom section 524 to a primary portion of the housing 502, a U-shaped latch 528 is formed on a wall 508 and a projection 530 is formed on a side wall 532 of the bottom section 524 (see FIG. 23). Other mechanisms for detachably mounting and securing the bottom section 524 to the primary portion of the housing 502 are also envisioned and within the scope of the invention.

A mechanism 534 for forming a roll of waste enclosed by lining material 578 is arranged in connection with the housing 502. The mechanism 534 comprises a take-up roll 536 rotatably mounted in the bottom section 524 and a rotation device 538 for rotating the take-up roll 536. The rotation device 538 generally comprises a manually actuatable handle 540 and a transmission mechanism 542 for converting the downward movement of the handle 540 into rotational movement of the take-up roll 536 while preventing upward movement of the handle 540 from being converted into rotational movement of the take-up roll 536.

There are various different forms of the take-up roll 536 which may be used in the invention. Generally though, the take-up roll 536 is substantially cylindrical and includes one or more protruding nails 544 which penetrate the lining material 578 and thereby attach the lining material 578 to the take-up roll 536. Alternative take-up rolls are described above.

The transmission mechanism 542 comprises a lever 546 movable in a slot 548 formed in the front wall 504 of the housing 502 and which pivots about a pivot axis to thereby define a first lever arm 550 and a second lever arm 552. The handle 540 is attached to or formed integral with the first lever arm 550. The second lever arm 552 includes a series of teeth 554 on an outer arcuate surface which may be formed as a rack gear attached to the lever 546 or formed integral therewith. Optionally, stops may be provided to limit the movement of the first lever arm 550 in the slot 548 and a return spring (not shown in this embodiment) may be provided to return the lever 546 to a position in which the first lever arm 550 is at or near the top of the slot 548 (and the handle 540 is at an upper position ready for use). Other mechanisms for biasing the lever 546 to return to a position in which the handle 540 is at an upper position can also be applied in the invention.

The transmission mechanism 542 also includes a gear system or assembly 556 including at least one gear for converting the pivoting movement of the lever 546 into rotation of the take-up roll 536. The gear system 556 includes a one-way clutch gear 558 which transmits the movement of the second lever arm 552 into rotation of the take-up roll 536 only when the second lever arm 552 rotates toward the rear wall 506, which results when the handle 540 is pulled downward. When the handle 540 returns to its biased position, i.e., upward, and the lever 546 pivots in a counterclockwise direction, the gear system 556 does not transmit the pivoting movement of the lever 546 into rotation of the take-up roll 536.

To this end, the clutch gear 558 has a rotation pin mounted in elongate slots and is moved by the counterclockwise rotation of the lever 546 into an extreme position in the slots wherein it is in engagement with a second gear 560 (see FIG. 26). On the other hand, when the lever 546 rotates in clockwise direction, the clutch gear 558 is moved to the other extreme position in the slots in which it is not in engagement with the second gear 560.

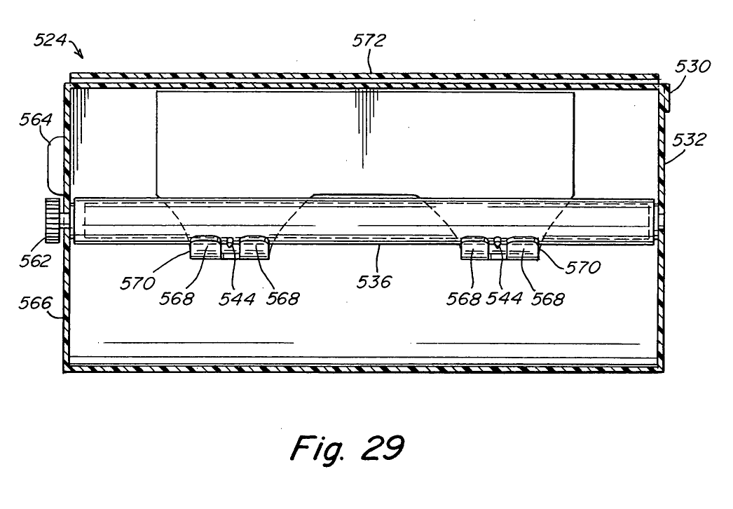

The gear system 556 also includes a third gear 562 in continual engagement with the second gear 560 (when the bottom section 524 is attached to the primary portion of the housing 502). The third gear 562 is connected to the take-up roll 536 as shown in FIG. 29, so that when the bottom section 524 is engaged with the housing 502, the third gear 562 engages with the second gear 560. To aid in the proper engagement of the bottom section 524 to the first portion of the housing 502, projections 564 are formed on the side wall 566 of the bottom section 524 (see FIG. 29). Other alignment techniques can be used in the invention.

The lever 546 and gear system 556 are housed in a compartment in the housing 502 defined between the side wall 508 and an intermediate wall 574. Intermediate wall 574 includes an aperture through which the third gear 562 passes to engage with the second gear 560. To provide access to the compartment between the side wall 508 and intermediate wall 574, the side wall 508 can be detachably connected to a remaining portion of the housing 502, e.g., via a snap fit.

The take-up roll 536 is connected to the third gear 562 at one axial end and at an opposite axial end, the take-up roll 536 is mounted or fixed to a side wall 532 of the bottom section 524 of the housing 502 (see FIG. 29).

Bottom section 524 also includes spring fingers 568 to facilitate retention of waste being rolled onto the take-up roll 536 to form the roll of enclosed waste and lining material. Spring fingers 568 are attached to a rear wall of the bottom section 524 and biased to press the lining material 578 against the take-up roll 536. In the illustrated embodiment, there are two finger members 570, each including two fingers 568, although any number of finger members 570 and fingers 568 can be used in the invention.

The fingers 568 guide the lining material 578 from a new roll 576 into engagement with the protruding nail(s) 544 on the take-up roll 536, and also serve to compact the waste between the windings of the lining material 578.

The bottom section 524 also includes a hinged cover 572 which is open when the bottom section 524 is in engagement with the remaining portion of the housing 502 so that the lining material and waste passes into the lower compartment 526. The cover 572 is closed when the bottom section 524 is removed and discarded so that the roll of lining material and enclosed waste in the bottom section 524 is secured therein and the waste cannot fall out.

The features in the embodiments described above with reference to FIGS. 1-22 can also be applied in the embodiment of FIGS. 23-29 and vice versa, to the extent possible.

To prepare the system 500 for use, the housing 502 is mounted to a wall and the bottom section 524 with a take-up roll 536 is attached to the housing 502 (with the hinged cover 572 in an open position), such that the third gear 562 is engaged with the second gear 560. The door 522 is opened and a roll of lining material 576 is placed into the compartment 514 and unwound slightly until a forward edge portion can be guided through the dispensing slot 520 between the upper shelf 516 and the rear wall 506 of the housing 502. The lining material 578 is pushed downward through dispensing slot 520 until it buckles slightly indicating that the forward edge is sandwiched at a nip between the fingers 568 and the take-up roll 536. The handle 540 is then depressed to cause rotation of the take-up roll 536 and the nail(s) 544 to penetrate into the lining material 578. Once each nail 544 penetrates into the lining material 578, subsequent rotation of the take-up roll 536 will cause a length of lining material 578 to wind about the take-up roll 536.

The doors 522 can be closed and the system 500 is ready for use. If an optional shelf protector is used, it may be attached to the lower surface of the upper shelf 516 by removing the coating material from the adhesive and adhering it to the lower surface of the upper shelf 516.

In one exemplifying use, waste, such as a syringe and a glove, is placed into the slot 512 and pushed to the end of the slot 512 along the lower shelf 518 to fall out of the slot 512 at a rear of the housing 502 into engagement with the lining material 578 from the roll 576. The handle 540 is then pushed downward causing rotation of the second lever arm 552 in the counterclockwise direction and attendant movement of the rotation pin of the first gear 558 in the elongate slots until it engages the second gear 560. Continued rotation of the second lever arm 552 causes rotation of the first gear 558 which in turn causes rotation of the now-engaged second gear 560 and the take-up roll 536 connected thereto. Rotation of the take-up roll 536 exerts a pulling action on the lining material 578 causing the waste to be urged toward the nip between the fingers 568 and the take-up roll 536, e.g., via frictional engagement between a fabric paper side of the lining material 578 and the waste. The fingers 568 press the waste against a winding of lining material 578 previously wound onto the take-up roll 536. As such, the waste is enclosed between successive windings of the lining material 578.

Once the compartment 526 has a full roll of enclosed waste and lining material, the entire bottom section 524 is disengaged from the primary portion of the housing 502 and the hinged cover 572 is closed to send in any contamination during disposal or prior to recycling. The lining material 578 may contain horizontal perforations to enable it to be torn after a full roll of enclosed waste and lining material is formed and used to form another roll of enclosed waste and lining material once a new bottom section 524 is attached to the housing 502. In this case, the door 522 would be opened and the lining material 578 advanced to the nip between the take-up roll 536 and fingers 568 and attached to the take-up roll 536 in the same manner as described above.

The full bottom section 524 may either be disposed of, when the bottom section 524 is designed to be disposable, or if not disposable, picked-up and brought or delivered to a processing facility in which the roll of enclosed waste and lining material is removed. The bottom section 524 would also be cleaned. Thus, the bottom section 524 could either be entirely disposable or reusable with only the take-up roll 536 requiring replacement.

Thus, the foregoing constructions of waste disposal systems are extremely suitable for wall-mounting and for receiving and enclosing waste products, in particular medical waste products. The waste disposal systems can be beneficially used as sharps container in which potentially infectious medical waste products are enclosed in a lining without any potential for causing injury during handling of the enclosed waste products when the lining with enclosed waste products is removed from the container. Diapers and other similar odorous objects can also be advantageously disposed of using the wall-mounted waste disposal systems described above.

It should be clear that various modifications and alterations can be made within the scope of the present invention. Also, various features of one embodiment can be combined with features of other embodiments, consistent with proper operation thereof, within the scope of the present invention.

1. A waste disposal system, comprising:
a housing defining a waste receptacle, said housing having a slot into which waste is placed;
a first roll of lining material; and
roll forming means for forming a roll of enclosed waste and said lining material with the waste being enclosed between windings of said lining material.

2. The system of claim 1, wherein said slot divides said housing into an upper compartment and a lower compartment, said first roll being arranged in said upper compartment and said roll of enclosed waste and lining material being formed in said lower compartment.

3. The system of claim 1, wherein said housing includes an upper shelf defining an upper wall of said slot, said upper shelf being arranged to support said first roll.

4. The system of claim 3, wherein said housing further includes a rear wall, said upper shelf being spaced from said rear wall and defining a dispensing slot with said rear wall through which the lining material from said first roll passes.

5. The system of claim 1, wherein said slot is downwardly and inwardly oriented.

6. The system of claim 1, further comprising a second roll of lining material, said first and second rolls being arranged to unwind such that waste is sandwiched between the lining material from said first roll and the lining material from said second roll.

7. The system of claim 6, further comprising mounting means for rotatably mounting said second roll on said housing.

8. The system of claim 7, wherein said mounting means comprise brackets extending outward from said housing.

9. The system of claim 8, wherein said mounting means are arranged to rotatable mount said second roll below said slot such that said second roll passes over a lower surface defining said slot as it unwinds.

10. The system of claim 6, wherein the lining material of said second roll passes through said slot, the lining material of said first roll having a width larger than a width of said slot such that waste at edges of said slot is retained by the lining material of said first roll.

11. The system of claim 6, wherein the lining material of said first roll is paper fabric and the lining material of said second roll is plastic film.

12. The system of claim 6, wherein said housing further includes a first dispensing compartment for housing said first roll and a second dispensing compartment for housing said second roll.

13. The system of claim 12, wherein said second dispensing compartment is formed below said slot.

14. The system of claim 12, further comprising first and second doors for enabling selective access to said first and second dispensing compartments respectively.

15. The system of claim 12, wherein said slot is defined by a lower shelf, an upper shelf opposite said lower shelf and opposed lateral walls, said first dispensing compartment being arranged above said upper shelf, said second dispensing compartment being arranged below said lower shelf.

16. The system of claim 15, wherein said lateral walls project inward into said slot to define a lower dispensing passage above said lower shelf through which the lining material from said lower roll passes and an upper dispensing passage below said upper shelf through which the lining material from said upper roll passes, said lateral walls reducing the width of a waste-receiving portion of said slot.

17. The system of claim 1, wherein said roll forming means comprise a take-up roll rotatably mounted in said housing and rotation means for rotating said take-up roll.

18. The system of claim 17, wherein said take-up roll comprises a male or female coupling connection and said rotation means comprise a member having a complementary male or female coupling.

19. The system of claim 17, wherein said take-up roll has a first axial end releasably connected to said rotation means and a second axial end, further comprising retaining means for releasably retaining said second axial end of said take-up roll.

20. The system of claim 19, wherein said retaining means comprise a spring-loaded release mechanism having a projection adapted to engage with said second axial end of said take-up roll.

21. The system of claim 20, wherein said release mechanism comprises a substantially cylindrical main body part arranged in said housing and having a knob at an outer end and said projection at said inner end, said main body part having a flange for limiting axial movement of said main body part, said release mechanism further comprising a spring for biasing said projection inward into engagement with said second axial end of said take-up roll.

22. The system of claim 20, wherein said release mechanism comprises a locking device for preventing disengagement of said projection from said second axial end of said take-up roll.

23. The system of claim 22, further comprising a door leading to said lower compartment, said locking device comprising an annular groove formed in said release mechanism and a lock out arm arranged to be situated in said groove when said is closed and out of said groove when said door is open.

24. The system of claim 19, further comprising guide means for guiding placement of said second axial end of said take-up roll into engagement with said projection, said guide means comprising guide ramps and a locating rim formed on said housing proximate said retaining means.

25. The system of claim 19, wherein said retaining means comprise a spring arm connected to said housing and a biasing member having a projection adapted to engage with said second axial end of said take-up roll and arranged to be biased by said hinge arm into engagement with said take-up roll.

26. The system of claim 17, wherein said rotation means comprise a manually actuatable handle and a transmission mechanism for converting the downward movement of said handle into rotational movement of said take-up roll while preventing upward movement of said handle from being converted into rotational movement of said take-up roll.

27. The system of claim 26, wherein said transmission mechanism comprises a lever pivotable about a pivot axis upon manual actuation of said handle and at least one gear engaging with said lever for enabling the conversion of the manual actuation of said handle into rotation of said take-up roll.

28. The system of claim 27, further comprising movement limiting means for limiting movement of said lever and said handle.

29. The system of claim 27, wherein said lever has teeth formed on an outer surface thereof in engagement with said at least one gear.

30. The system of claim 27, wherein said at least one gear is part of a gear assembly constructed to convert movement of said handle in a downward direction to rotational movement of said take-up roll and prevent movement of said handle in an upward direction from being converted to rotational movement of said take-up roll.

31. The system of claim 27, wherein said transmission mechanism further comprises a mounting projection coupled to said at least one gear and arranged to rotationally engage with said take-up roll.

32. The system of claim 26, further comprising a cover plate for defining a compartment with said housing, said transmission mechanism being at least partially arranged in said compartment.

33. The system of claim 17, wherein said rotation means comprise a motor coupled to said housing and having an output shaft rotation upon actuation of said motor, a switch for actuating said motor and a coupling connected to said output shaft and to said take-up roll.

34. The system of claim 33, wherein said coupling has a male or female coupling connection and said take-up roll has a complementary male or female coupling.

35. The system of claim 17, wherein said take-up roll comprises attachment means for enabling attachment of the lining material of said first roll to said take-up roll.

36. The system of claim 35, wherein said attachment means comprise a single section of adhesive extending over a major part of an outer surface of said take-up roll.

37. The system of claim 35, wherein said attachment means comprise a plurality of discrete circular bands of adhesive each extending at a different axial location around an outer surface of said take-up roll.

38. The system of claim 36, wherein said attachment means comprise an axially extending slot allowing a full width of the lining material to pass therethrough.

39. The system of claim 36, wherein said attachment means comprise at least one barb arranged on an outer surface of said take-up roll.

40. The system of claim 17, wherein said take-up roll comprises a tubular sleeve and first and second plastic inserts separable from said sleeve and adapted to mount said sleeve in said housing.

41. The system of claim 40, wherein said sleeve includes attachment means for enabling attachment of the lining material of said first roll to said sleeve.

42. The system of claim 17, wherein said take-up roll comprises a disposable sleeve including attachment means for enabling attachment of the lining material of the first roll to said sleeve and a reusable core adapted to mount said sleeve in said housing, said sleeve being arranged to slide onto and off of said core.

43. The system of claim 17, wherein said take-up roll includes at least one protruding nail adapted to penetrate the lining material and thereby attach the lining material to said take-up roll.

44. The system of claim 1, wherein the lining material of said first roll includes perforation lines to enable tearing of the lining material.

45. The system of claim 1, further comprising a disposable shelf protector for covering an upper or lower surface of said slot.

46. The system of claim 45, wherein said shelf protector comprises adhesive for attaching said shelf protector to said housing.

47. The system of claim 1, further comprising mounting means for mounting said housing to a wall or other vertical surface.

48. The system of claim 47, wherein said mounting means comprise at least one suction cup fixed to said housing.

49. The system of claim 1, wherein said housing includes a detachable bottom section defining a waste-receiving compartment.

50. The system of claim 49, wherein said roll forming means comprise a take-up roll rotatably mounted in said bottom section and rotation means for rotating said take-up roll.

51. The system of claim 50, wherein said rotation means comprise a handle connected to said housing and transmission means for converting downward movement of said handle into rotational movement of said take-up roll while preventing upward movement of said handle from being converted into rotational movement of said take-up roll.

52. The system of claim 51, wherein said transmission means comprises a lever pivotable about a pivot axis upon manual actuation of said handle and at least one gear engaging with said lever for enabling the conversion of the manual actuation of said handle into rotation of said take-up roll.

53. The system of claim 52, wherein said at least one gear is part of a gear assembly constructed to convert movement of said handle in a downward direction to rotational movement of said take-up roll and prevent movement of said handle in an upward direction from being converted to rotational movement of said take-up roll.

54. The system of claim 53, wherein gear assembly includes a gear connected to one end of said take-up roll.

55. The system of claim 50, wherein said bottom section further includes spring fingers for facilitating retention of waste being rolled onto said take-up roll.

56. The system of claim 49, wherein said bottom section includes a hinged cover which is open when said bottom section is in engagement with a remaining part of said housing and closed when said bottom section is detached from said remaining part of said housing.
